(19) United States
(12) Patent Application Publication
(10) Pub. No.: US 2026/0120194 A1
(43) Pub. Date: Apr. 30, 2026

(54) AUTONOMOUS MEDICAL CODING SYSTEM

(71) Applicant: Oracle International Corporation, Redwood Shores, CA (US)

(72) Inventors: Dmytro Shamatrin, Orlando, FL (US); Maxim Romanenko, Altamonte Springs, FL (US); Ilia Gilderman, Seattle, WA (US); Harshit K. Kalley, Belmont, CA (US); Nathan Smalley, Los Angeles, CA (US); Qingyu Zhang, Santa Clara, CA (US)

(73) Assignee: Oracle International Corporation, Redwood Shores, CA (US)

(21) Appl. No.: 19/236,344

(22) Filed: Jun. 12, 2025

Related U.S. Application Data
(60) Provisional application No. 63/712,219, filed on Oct. 25, 2024.

(51) Int. Cl. G06Q 40/08; G16H 10/60; G16H 15/00

(52) U.S. Cl. CPC G06Q 40/08; G16H 10/60; G16H 15/00

(57) ABSTRACT

Techniques for autonomous assignment of medical codes are disclosed. A natural-language health record is processed, to identify a portion of an extracted text. By applying a binary classification on the portion, a codability of the portion is identified. In response to a positive codability, two or more codes are assigned to the portion, by applying a multi-label classification to the portion. By applying a probability model, a first probability score indicative of a probability of a combination of the two or more codes being assigned to a single encounter is determined. By applying a language model, a second probability score indicative of the two or more codes assigned to the health record being correct is determined. A final probability score is assigned. In response to the final probability score being higher than a threshold, generation of an insurance record is caused, based on the two or more codes.

### CROSS REFERENCE TO RELATED APPLICATIONS

This application claims the benefit of U.S. Provisional Ser. No. 63/712,219 filed on Oct. 25, 2024. The entire disclosure of the aforementioned application is incorporated by reference herein in its entirety for all purposes.

### BACKGROUND

Medical coding is essential in healthcare institutions, e.g., for receiving reimbursement for medical costs. For example, during a patient encounter, an electronic health record (EHR) is generated. A medical coder assigns one or more medical codes to the EHR, which are then used to seek reimbursement from insurance carriers. Medical coding is a time-consuming task, where the medical coder has to manually review health records generated by a healthcare provider and assign one or more medical codes based on such records.

### BRIEF SUMMARY

In various embodiments, a non-transitory computer-readable medium includes instructions that when executed by one or more processors, cause the one or more processors to perform a set of operations including: accessing a natural-language health record that is generated based on an encounter; processing the natural-language health record using a natural language processing model, to identify a portion of an extracted text from the natural-language health record; generating, by applying a binary classification on the portion of the extracted text, a codability of the portion of the extracted text, wherein the codability of the portion of the extracted text is based on whether or an extent to which the portion of the extracted text includes information for assignment of one or more codes; in response to a positive codability of the portion of the extracted text, assigning two or more codes to the portion of the extracted text by applying a multi-label classification by a machine learning model to the portion of the extracted text; determining, by applying a probability model, a first probability score indicative of a probability of a combination of the two or more codes being assigned to a single encounter; determining, by applying a language model, a second probability score that is indicative of the two or more codes assigned to the health record being correct; assigning, based at least in part on the first probability score and the second probability score, a final probability score to the two or more codes; and in response to the final probability score being higher than a threshold, causing generation of an insurance record based at least in part on the two or more codes.

In an example, to determine the first probability score, the probability model (i) takes into account the assigned two or more codes, and (i) does not take into account the extracted text. In an example, to determine the second probability score, the language model takes into account (i) the assigned two or more codes and (ii) one or both of (a) the extracted text and the (b) health record. In an example, the first probability score is a conditional probability of a first one of the two or more codes being assigned to the single encounter, given that a second one of the two or more codes is assigned to the single encounter. In an example, the probability model is a one of a TF-IDF (term frequency-inverse document frequency) based model, a KNN (k-nearest neighbors) model, or a PMI (probability mutual information) model.

In an example, causing generation of the insurance record comprises: generating one or more billable codes, based on the one or more codes; and causing generation of the insurance record, based at least in part on the one or more billable codes. In an example, the encounter is a first encounter, the health record is a first health record, the two or more codes are first two or more codes, the final probability score is a first final probability score, the insurance record is a first insurance record, and wherein the set of operations further include: accessing a second natural-language health record that is generated based on a second encounter; assigning second one or more codes to the second natural-language health record, by applying a multi-label classification by the machine learning model to another portion of an extracted text of the second natural-language health record; assigning a second final probability score to the second one or more codes; in response to the second final probability score lower than the threshold, receiving a correction of at least one of the second one or more codes; and causing generation of a second insurance record based at least in part on the corrected second one or more codes. In an example, the set of operations further include: generating training data, based at least in part on the portion of the extracted text of the second natural-language health record and the correction of at least one of the second one or more codes; and training the machine learning model using the training data. In an example, the set of operations further include: causing display of an identification of the patient encounter and/or the health record, the two or more codes assigned to the health record, and the final probability score. In an example, accessing the natural-language health record comprises: pulling the health record from an electronic health record system; or receiving a push of the health record from the electronic health record system. In an example, preprocessing the natural-language health record comprises: removing personally identifiable information, such that the extracted natural-language text from the health record lacks any personally identifiable information. In an example, causing generation of the insurance record comprises: grouping the two or more codes into a single group code; generating at least one billable code, based at least in part on the single group code; and causing generation of the insurance record, based at least in part on the at least one billable code.

In various embodiments, a method comprises: accessing a natural-language health record that is generated based on an encounter; processing the natural-language health record using a natural language processing model, to identify a portion of an extracted text from the natural-language health record; generating, by applying a binary classification on the portion of the extracted text, a codability of the portion of the extracted text, wherein the codability of the portion of the extracted text is based on whether or an extent to which the portion of the extracted text includes information for assignment of one or more codes; in response to a positive codability of the portion of the extracted text, assigning two or more codes to the portion of the extracted text by applying a multi-label classification by a machine learning model to the portion of the extracted text; determining, by applying a probability model, a first probability score indicative of a probability of a combination of the two or more codes being assigned to a single encounter; determining, by applying a language model, a second probability score that is indicative of the two or more codes assigned to the health record being correct; assigning, based at least in part on the first probability score and the second probability score, a final probability score to the two or more codes; and in response to the final probability score being higher than a threshold, causing generation of an insurance record based at least in part on the two or more codes. In an example, to determine the first probability score, the probability model (i) takes into account the assigned two or more codes, and (i) does not take into account the extracted text. In an example, to determine the second probability score, the language model takes into account (i) the assigned two or more codes and (ii) one or both of (a) the extracted text and the (b) health record. In an example, the probability model is a one of a TF-IDF (term frequency-inverse document frequency) based model, a KNN (k-nearest neighbors) model, or a PMI (probability mutual information) model. In an example, causing generation of the insurance record comprises: grouping the two or more codes into a single group code; generating at least one billable code, based at least in part on the single group code; and causing generation of the insurance record, based at least in part on the at least one billable code.

In various embodiments, a system comprises: one or more processors; and one or more non-transitory computer-readable media storing instructions, which, when executed by the system, cause the system to perform operations including: accessing a natural-language health record that is generated based on an encounter; processing the natural-language health record using a natural language processing model, to identify a portion of an extracted text from the natural-language health record; generating, by applying a binary classification on the portion of the extracted text, a codability of the portion of the extracted text, wherein the codability of the portion of the extracted text is based on whether or an extent to which the portion of the extracted text includes information for assignment of one or more codes; in response to a positive codability of the portion of the extracted text, assigning two or more codes to the portion of the extracted text by applying a multi-label classification by a machine learning model to the portion of the extracted text; determining, by applying a probability model, a first probability score indicative of a probability of a combination of the two or more codes being assigned to a single encounter; determining, by applying a language model, a second probability score that is indicative of the two or more codes assigned to the health record being correct; assigning, based at least in part on the first probability score and the second probability score, a final probability score to the two or more codes; and in response to the final probability score being higher than a threshold, causing generation of an insurance record based at least in part on the two or more codes. In an example, to determine the first probability score, the probability model (i) takes into account the assigned two or more codes, and (i) does not take into account the extracted text; and to determine the second probability score, the language model takes into account (i) the assigned two or more codes and (ii) one or both of (a) the extracted text and the (b) health record. In an example, the first probability score is a conditional probability of a first one of the two or more codes being assigned to the single encounter, given that a second one of the two or more codes is assigned to the single encounter.

In some embodiments, a system comprises: one or more processors; and one or more non-transitory computer-readable media storing instructions, which, when executed by the system, cause the system to perform part or all of one or more methods disclosed herein.

In some embodiments, a system is provided that includes one or more data processors and a non-transitory computer-readable storage medium containing instructions which, when executed on the one or more data processors, cause the one or more data processors to perform part or all of one or more methods disclosed herein.

In other embodiments, a computer-program product is provided that is tangibly embodied in a non-transitory machine-readable storage medium and that includes instructions configured to cause one or more data processors to perform part or all of one or more methods disclosed herein.

Cloud services, microservices, or other machine-hosted services may be offered that perform part or all of one or more methods disclosed herein. The machine-hosted services may be provided by a single machine, by a cluster of machines, or otherwise distributed across machines. The one or more machines may be configured to send and receive data, which may include instructions for performing the methods or results of performing the methods, via an application programming interface (API) or any other communication protocol.

In various embodiments, part or all of one or more methods disclosed herein may be performed by stored instructions such as a software application, computer program, or other software package installed in memory or other storage of a computing platform, such as an operating system, which provides access to physical or virtual computing resources. The operating system may provide access to physical or virtual resources of a mobile computing device, a laptop computing device, a desktop computing device, a server computing device, a container in a virtual machine on a computing device, or any other computing environment configured to execute stored instructions.

As used herein, the terms “first,” “second,” “third,” “fourth,” etc. are used as naming conventions to refer to separate items in a set of items. These naming conventions do not imply ordering unless such ordering is explicitly noted using language specific to ordering, such as “before” or “after,” or unless such ordering is required to attain the expressly recited functionality, such as generating an item and later accessing the generated item.

The techniques described above and below may be implemented in a number of ways and in a number of contexts. Several example implementations and contexts are provided with reference to the following figures, as described below in more detail. However, the following implementations and contexts are but a few of many.

### BRIEF DESCRIPTION OF THE DRAWINGS

Various embodiments are described hereinafter with reference to the figures. It should be noted that the figures are not drawn to scale and that the elements of similar structures or functions are represented by like reference numerals throughout the figures. It should also be noted that the figures are only intended to facilitate the description of the embodiments. They are not intended as an exhaustive description of the disclosure or as a limitation on the scope of the disclosure.

FIG. 1A illustrates a block diagram of an autonomous medical coding system, which autonomously assigns medical codes based on electronic health records.

FIG. 1B illustrates a validation operation on assigned codes generated by a model backend service and/or an encoder system of an autonomous medical coding system.

FIG. 2 illustrates a flow diagram illustrating operations of an autonomous medical coding system.

FIG. 3 illustrates a flow diagram illustrating data flow within an autonomous medical coding system.

FIG. 4A illustrates a method for autonomously assigning codes to a patient encounter.

FIG. 4B illustrates a method for autonomously assigning and validating codes to a patient encounter.

FIG. 5 depicts a simplified diagram of a distributed system for implementing certain aspects.

FIG. 6 is a simplified block diagram of one or more components of a system environment by which services provided by one or more components of an embodiment system may be offered as cloud services, in accordance with certain aspects.

FIG. 7 illustrates an example computer system that may be used to implement certain aspects.

### DETAILED DESCRIPTION

Usage of medical coding is ubiquitous in healthcare settings, e.g., for receiving reimbursement for medical costs. For example, once a medical care professional interacts with a patient during a patent encounter, the medical care professional summarizes the encounter in writing, to generate an electronic health record (EHR). In another example, an EHR may be generated, based on a patient visit to a laboratory or a medical diagnostic center. A medical coder assigns one or more medical codes to the EHR. Medical coding is a process by which healthcare diagnosis, procedures, medical services, and equipment are transformed or mapped into universal medical alphanumeric codes. Such medical codes are taken from medical record documentation (e.g., the EHR), such as transcription of notes or after-visit summary captured by medical care professionals, laboratory and radiologic results, etc. For example, medical coding professionals (also referred to as medical coders) review such medical record documentation, and assign appropriate medical codes for encounters between a patient and one or more medical care professionals. The medical codes are then used by the medical coding professionals and/or medical coding institutions (such as by physicians and/or hospitals) for reimbursement purposes. A medical claim includes one or more such medical codes, and the medical claim is submitted to medical insurance carriers for reimbursement. For example, medical coding occurs when a patient visits a healthcare provider. The healthcare provider interacts with the patient and treats the patient and documents the visit. A medical coder later reviews the document, and assigns one or more medical codes for the visit. The healthcare provider (or a facility in which the healthcare provider works) then submits a claim seeking reimbursement from the insurance carriers for the patient encounter, where the claim includes the medical codes.

Medical coding is a time-consuming task, where a medical coder has to manually review medical documents (such as EHR) for patient encounters and assign one or more medical codes based on such medical documents. Accordingly, disclosed herein are techniques for autonomous assignment of medical codes, e.g., using artificial intelligence (AI) and machine learning (ML) models. For example, an autonomous medical coding system ingests EHRs from an EHR system (e.g., either using a push interface or a pull interface), and autonomously assign one or more medical codes for each such EHRs.

The disclosed techniques are applicable for different types of patient encounters, such as outpatient encounters (e.g., where a patient receives care without being admitted to a hospital or a healthcare facility overnight) and inpatient encounters (e.g., where a patient is admitted to a hospital or a healthcare facility for at least one overnight stay). Similarly, the disclosed techniques are applicable to different types of medical coding, such as professional coding (e.g., for services rendered by healthcare professionals including doctors, nurse practitioners, and physician assistants) and facility coding (e.g., for services and resources provided by the hospital or other healthcare facilities).

The autonomous medical coding system described herein includes a data ingestion system that receives health records (such as EHRs) from an EHR system (where the EHR system is described below in detail), e.g., using either a data pull interface or a data push interface, and stores the received EHRs to a data storage system. The EHRs are stored as raw data within the data storage system.

The autonomous medical coding system further comprises a text preprocessing system that pre-processes the raw data stored within the data storage system and generates pre-processed data. For example, the text preprocessing system performs text extraction, de-identification of confidential patient information, and/or text cleanup. In an example, the text preprocessing system implements a text data exchange protocol, which enables the text preprocessing system to operate with any type of formats for the EHRs. The text data exchange protocol feeds the raw data to the text preprocessing system and stores the resultant preprocessed data to the data storage system. In an example, the text preprocessing system implements a text extraction service that extracts text from ingested documents (such as ingested raw data or EHR). In an example, the text extraction service uses optical character recognition (OCR) techniques, or other techniques to extract text from the raw data.

In an example, the text preprocessing system further implements a de-identification service that identifies and removes personally identifiable information (PII) from the extracted text. Thus, the preprocessed data stored in the data storage system may not include personally identifiable information of the patient. In an example, the text preprocessing system further implements a text cleanup service that performs additional cleanup of the preprocessed data, e.g., to remove any unnecessary information and markup text, such as page numbers, barcode values from headers, hospital seals, etc.

The autonomous medical coding system further includes a natural language processing (NLP) system that receives the pre-processed data (e.g., from the data storage system, or directly from the text preprocessing system). The NLP system processes the pre-processed data, such as performs natural language processing on the pre-processed data, e.g., to find potential points of interest within the pre-processed data. For example, the NLP system performs binary classification on the extracted text of the pre-processed data, e.g., to determine whether the extracted text has codable medical information (e.g., to determine a codability of the extracted text). For example, the NLP system identifies one or more portions of the pre-processed data, which includes information that can be used for (or is pertinent to) medical coding.

The NLP system implements a model data exchange protocol, which is a protocol designed to exchange data between the NLP system and a ML model backend service. The model data exchange protocol facilitates use of any type of pluggable model backend with the NLP system. In an example, the model data exchange protocol defines a way of interaction between the NLP system and the model backends.

In an example, the NLP system further implements a language model, such as a large language model (LLM), which, for example, performs text chunking, e.g., to find potential points of interest within the extracted text of the preprocessed data. For example, the potential points of interest are sections of the text relevant to assignment of medical codes, as also described above.

In an example, the autonomous medical coding system further includes a model backend service. The model backend service hosts one or more artificial intelligence (AI) models and/or ML models for autonomous coding, grouping or bundling of codes, text cleanup, classification, text generation, and/or other tasks.

The model backend service receives extracted and processed texts from the NLP system. In an example, the model backend service comprises one or more coding models and/or one or more grouping models.

In an example, the coding models are configured to assign one or more codes to an encounter between a patient and one or more health care professionals. For example, based on the text parsed, chunked, classified, and/or highlighted by the NLP system, the coding models assign codes to the encounter results, such as codes associated with the encounter. In an example, coding models for coding and/or evaluation are represented by multi-label classification models, which are based on LLMs pre-trained on large medical corpora and/or generative AI models, pre-trained for general purpose tasks. In an example, customized models may also be used, wherein a customized model perform feature extraction (e.g., extracts features from given texts parts containing medical coding information, as generated by the NLP system) and/or performs reasoning detection (e.g., aims to find within the extracted text reasoning for assigning a medical code, generate code reasoning, and/or provide a reference to particular place of document with information relevant to the code).

In an example, the one or more grouping models may be used for grouping two or more of the medical components of the EHR to form a bundled or group code. For example, bundling or grouping aims to group two or more medical components and assigns a single code to the group. Grouping aims to streamline billing and reimbursement and avoid overpayment or double counting for related services. For example, during a single patient encounter, multiple diagnosis and/or procedures may be performed, where such multiple diagnosis and/or procedures may be grouped or bundled under a single medical code, as described below in further detail.

In an example, the grouping models may be at least in part or fully integrated with the coding models. In an example, the grouping models include LLM models and/or statistical models. LLM models are used for feature extraction and input documents analysis alongside codes from coding models, to predict grouper codes in the provided nomenclature and/or ontology. In an example, statistical and classification models may be used to generate groupers based on probability of some groupers, given the combination of the input codes.

In an example, the validation system implements a plurality of statistical and/or probabilistic models, and/or language models (such as LLMs) to predict a probability of the assigned codes being correct. An example of a statistical and/or probabilistic model used by the validation system includes a probability mutual information (PMI) model. The PMI model predicts a conditional probability of the event of code A, given a code B event occurred. In an example, the PMI model generates a probability of two or more codes to be predicted together for a single given patient encounter, and a probability of such two or more codes not to be predicted together for a single given patient encounter. The PMI model outputs a probability score, which may be a conditional probability of a first one of two or more codes being assigned to a single given encounter, given that remaining ones of the two or more codes are assigned to the single given encounter. Note that the health record and/or the extracted text, based on which the codes are assigned, are not used by the PMI model to generate the corresponding probability score.

Another example of a statistical and/or probabilistic model used by the validation system includes a TF-IDF (term frequency-inverse document frequency) based model. The TF-IDF is the product of two statistical term, term frequency and inverse document frequency. The TF-IDF is a measure of importance of a word (or in this case, a code) to a document in a collection or corpus, adjusted for the fact that some words appear more frequently in general. An adapted TF-IDF metric shows the probability and uniqueness of code combinations. In an example, the TF-IDF model receives two or more codes assigned to a health record of an encounter being validated, and outputs a corresponding probability score indicative of a correctness of a combination of the two or more assigned codes. In an example, the health record and/or the extracted text, based on which the codes are assigned, are not used by the TF-IDF model to generate the probability score (e.g., as the model relies on the codes, and not on documents from which the codes are derived, to generate the corresponding probability score).

Yet another example of a statistical and/or probabilistic model used by the validation system includes a KNN (k-nearest neighbors) model. The KNN model generates a statistical metric indicative of how often the assigned two or more codes appear together. In an example, the KNN model outputs a probability score, which may be indicative of a correctness of a combination of the two or more assigned codes. In an example, the health record and/or the extracted text, based on which the codes are assigned, are not used by the KNN model to generate the probability score (e.g., as the model relies on the assigned codes, and not on documents from which the codes are derived, to generate the corresponding probability score).

In an example, the validation system further implements a language model (such as a LLM). The language model receives the two or more codes assigned to the health record of the encounter being validated, as well as receives reference documents (such as raw data, pre-processed data, extracted text, and/or data generated by the NLP system and used by the model backend service to generate the coding results). Based on the reference documents, the language model generates a corresponding probability score of the assigned codes being correct.

In an example, the validation system further includes a scoring service that receives the probability scores, and generates a final probability score indicative of a probability of the assigned codes being correct. In an example, the scoring service relies on a voting-based system, where probability scores from multiple statistical and/or probabilistic model and the language model are combined to generate a final result. The scoring service may average (such as a weighted average) the probability scores from the various models, to arrive at the final probability score. The final probability score being higher than a threshold score implies that the assigned codes are correct. For example, if the final probability score is higher than a high threshold, the assigned codes are assumed to be correct and approved without human intervention or verification. If the final probability score is between the high threshold and a low threshold, validity of the codes is assumed to be questionable, and the codes are transmitted to a human coder for manual verification. If the final probability score is less than the low threshold, the codes are assumed to be incorrect, and the reference documents (such as raw data, pre-processed data, and/or data generated by the NLP system and used to generate the coding results) are provided to a human coder for re-coding. In an example, codes that are verified by human coders or recoded by human coders may be used to train one or more machine learning models of the autonomous medical coding system, as described below in further detail.

In an example, the autonomous medical coding system further includes a user interface (UI) system that includes a plurality of UIs. The UI system includes a results review and correction UI, which displays a final stage of medical coding. When codes are assigned and the system does its job, the user may approve or reject the coding results. In another example, approval could happen automatically, if the validation system approves the coding results.

The UI system further includes a browse cases/documents UI. In an example, this UI enables users to browse cases/coding results and other statistical information. The UI system includes an encoder UI, which displays results of the encoder system.

In an example, when the validation system flags coding results, grouping results, and/or final billable codes as possibly being erroneous, a medical coder views such error message through a corresponding UI, and works to correct the coding results, grouping results, and/or final billable codes as needed.

In an example, the autonomous medical coding system further includes a data augmentation system, which augments the coding results, the raw data, the preprocessed data, the training data, and/or other data generated by the system. For example, after such augmentation, the training data is used to train the coding models and/or the grouping models, as described below in further detail.

In an example, the autonomous medical coding system further includes a model training system, which includes a model training service. The model training service may be used for model training (such as continuous model training) of the coding models and/or the grouping models. The model training system also includes a dataset generation service, which generates training data usable to train the coding models and/or the grouping models, as also described below in further detail.

A technical challenge addressed by some embodiments of the invention relates to validation of assigned codes. For example, a traditional system (either manual or automatic code assignment system) may assign codes to a patient encounter. However, if desired, a coder may have to manually verify the validity of the assigned code, which is time consuming and prone to errors. A technical solution to such a technical challenge provided by some embodiments includes techniques that leverage on one or more probability and/or statistical metrics, as well as a language model, to verify a validity of the assigned code(s). In an example, a validation system generates a final probability score for one or more codes assigned to a patient encounter, based on probability scores from a plurality of probability and/or statistical models and at least one language model, to increase a confidence of the final result. For example, when two or more codes are assigned to a single encounter, a probability or statistical model examines the assigned codes, to generate a first probability score indicative of a probability of a combination of the two or more codes being assigned to a single encounter. Here, the probability or statistical model is agnostic to or ignores the actual health record(s) (based on which the codes were assigned) and focuses on the combination of the assigned codes. Furthermore, a language model examines the assigned codes, as well as the health record(s) (based on which the codes were assigned), to determine a second probability score that is indicative of the assigned codes to the patient encounter being correct. Thus, this dual approach of the at least two probability scores from the at least two different models (one that examines solely the assigned codes, and another that examiner the assigned codes and the associated health records) improves a versatility of the validation approach, and yields a more accurate final probability score. A final score is then generated, based on the individual scores form the probability or statistical model(s) and the language model.

Another technical challenge addressed by some embodiments of the invention relates to incompatibility of a coding system to different types of EHR systems. For example, a traditional coding system that works with a first EHR system has to be modified significantly to work with a second EHR system. A technical solution provided by some embodiments includes techniques that leverage on capabilities of a data ingestion system to pull health records from an EHR system, as well as to allow the EHR system to push health records to the data ingestion system. This enables a plug-and-play capability or interchangeability of EHR systems, as the autonomous medical coding system can work with any EHR system supporting such pull or push functionality.

Yet another technical challenge addressed by some embodiments of the invention relates to incompatibility in file types used for health records in various health care settings and/or in various EHR systems. A technical solution provided by some embodiments includes implementation of a text data exchange protocol, which can act on text files having any suitable format for health records. The text data exchange protocol defines a payload structure (e.g., a structure or format in which raw data is stored within the health record), and/or metadata associated with the health record and/or the raw data. The text data exchange protocol provides a way of representing input clinical documents and processed documents output.

A further technical challenge addressed by some embodiments of the invention relates to incompatibility in coding standards in various countries and/or regions using various types of coding standards. For example, the USA uses ICD-10-CM (Clinical Modification), which is a USA-specific modification of ICD-10 maintained by WHO (World Health Organization). In contrast, India typically uses standard ICD-10 from WHO, without USA-specific clinical modifications. A tradition coding system may not be compatible with different coding standards of different countries or regions. A technical solution provided by some embodiments includes implementation of a model data exchange protocol and/or an encoder data exchange protocol, which allows a model backend service and/or an encoder service of the autonomous medical coding system to be easily swapped and replaced by country specific services. These data exchange protocols enable usage of the autonomous medical coding system across different countries or regions, across different vendors, and/or across different disease nomenclatures. Numerous examples and configurations of the anomaly detection system are now described in further detail.

### Autonomous Medical Coding System

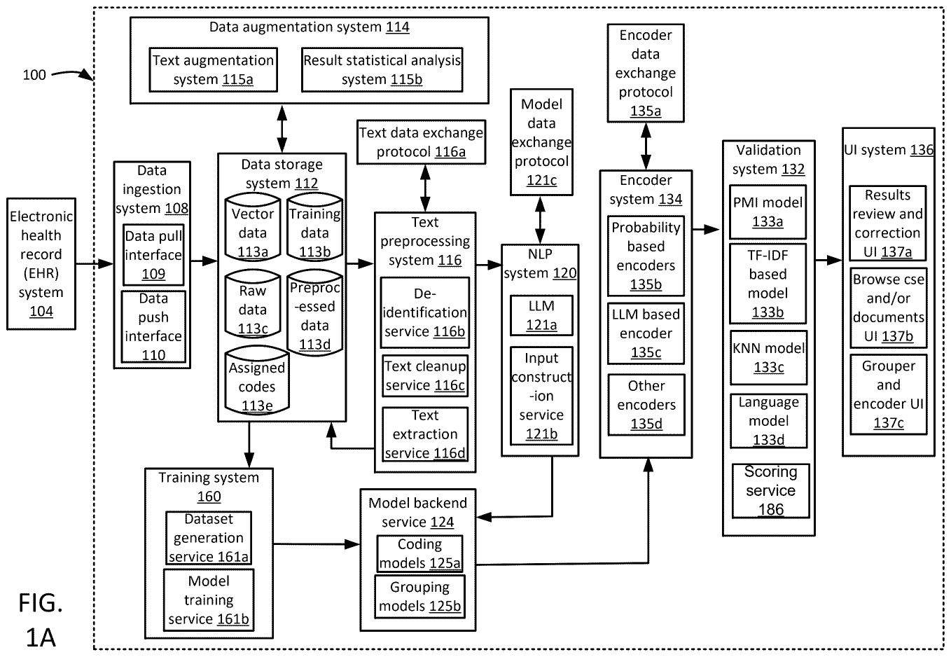

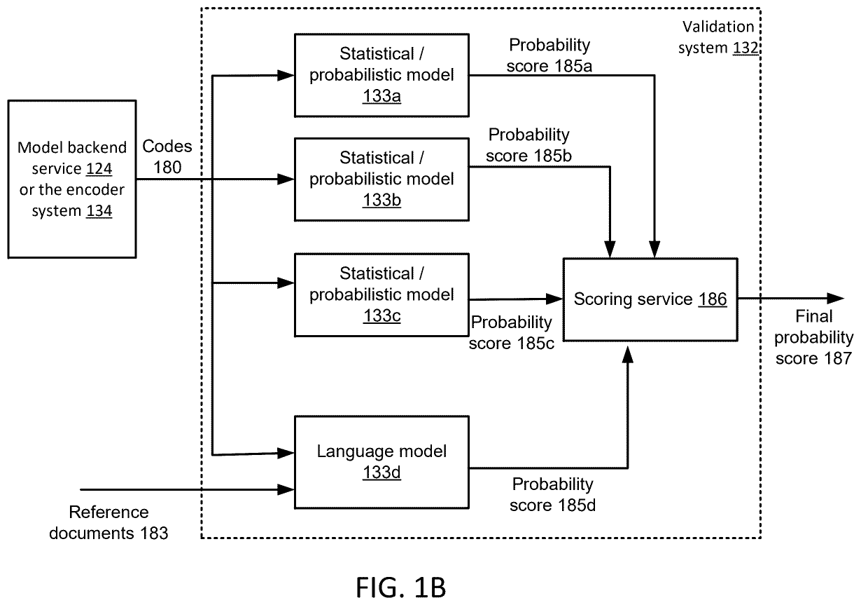

FIG. 1A illustrates a block diagram of an autonomous medical coding system 100 (also referred to herein as system 100), which autonomously assigns medical codes based on electronic health records. The autonomous medical coding system 100 communicates with an electronic health record (EHR) system 104 that provides EHRs to the autonomous medical coding system 100. Healthcare providers (such as doctors, nurses, technicians operating diagnostic equipment, and/or other healthcare professionals directly or indirectly interacting with patients during patient encounter events) provide EHRs to the EHR system 104, based on a patient encounter with the healthcare providers. The EHRs are in natural language, such as English or another natural language. The EHRs provided to the EHR system 104 includes medical record documentation, such as transcription of notes or after-visit summary captured by medical care professionals, laboratory and radiologic results, etc. Thus, for a patient encounter with a medical professional, EHR system 104 generates or otherwise receives a corresponding one or more EHRs. The autonomous medical coding system 100 receives such EHRs from the EHR system 104 and aims to assign corresponding medical codes for the patient encounter.

The autonomous medical coding system 100 includes a data ingestion system 108 configured to receive EHRs from the EHR system 104. In an example, the data ingestion system 108 pulls EHRs from the EHR system 104. For example, the data ingestion system 108 includes a data pull interface 109 for pulling EHR from the EHR system 104. Thus, the data ingestion system 108 actively downloads the EHR data from an EHR system 104.

In another example, the EHR system 104 pushes EHRs to the data ingestion system 108. For example, the data ingestion system 108 includes a data push interface 109 for receiving EHRs pushed by the EHR system 104 to the data ingestion system 108. Thus, the data push interface 109 allows a third-party EHR system 104 to upload EHRs to the data ingestion system 108.

Thus, in an example, the data ingestion system 104 may include appropriate plugin interfaces to pull or download data from a EHR system 104, and/or receive data pushed from the EHR system 104.

The autonomous medical coding system 100 further includes a data storage system 112. The data storage system 112 comprises one or more storage repositories for storing various types of data. For example, a first storage repository stores vector data 113a. For example, as described below in further detail, a text preprocessing system 116 and/or an NLP system 120 receives EHRs including patient inputs, and vectorizes the patient data. The vector data 113a includes such vectorized embedding of patient inputs. In another example, any vectorized embedding of data received from an appropriate document may be stored as vector data 113a within the data storage system 112.

In an example, the data storage system 112 comprises a second storage repository to store training data 113b. Training data 113b comprises data used for training one or more models of the autonomous medical coding system 100. For example, codes generated by the autonomous medical coding system 100 and manually validated or corrected by manual medical coders are stored as training data 113b within the data storage system 112 and used to train or retrain one or more ML models described below.

In an example, the data storage system 112 comprises a third storage repository to store raw data 113c. The raw data 113c comprises EHRs from the EHR system 104. In an example, the raw data 113c includes patient name, demographics, and/or other personal information identifying the patient, as well as medical records, patient visit summary, laboratory charges, diagnostic results, etc. Thus, the raw data 113c may include personally identifiable information (PII) and/or electronic protected health information (ePHI). Accordingly, in an example, the raw data 113c may be (such as has to be) compliant with health privacy regulation of a region or country in which the autonomous medical coding system 100 is to be deployed. For example, in the United States of America (USA), the raw data 113c may be Health Insurance Portability and Accountability Act (HIPAA) compliant. For example, access to the raw data 113c may be restricted and regulated. In an example, the raw data 113c may be encrypted and stored in one or more certified storage repositories, with restricted access. In an example, the text preprocessing system 116 can access the raw data 113c. Access to the raw data 113c may be restricted, such that no other components (or a limited number of components) of the autonomous medical coding system 100 may have access to the raw data 113c.

In an example, the data storage system 112 comprises a fourth storage repository to store preprocessed data 113d. For example, a text preprocessing system 116 (described below) processes raw data 113c, to generate the preprocessed data 113d. The preprocessed data 113d is later analyzed by the autonomous medical coding system 100, e.g., to assign corresponding medical codes.

The data storage system 112 comprises a fifth storage repository to store codes 113e assigned by the autonomous medical coding system 100. For example, the autonomous medical coding system 100 analyzes the preprocessed data 113d, to assign codes 113e, and the codes are stored in the data storage system 112 as assigned codes 113e.

The autonomous medical coding system 100 further comprises a text preprocessing system 116. The text preprocessing system 116 pre-processes raw data 113c stored within the data storage system 112, and generates pre-processed data 113d, as described above. For example, the text preprocessing system 116 performs text extraction, de-identification of confidential patient information, and/or text cleanup. In an example, the text preprocessing system 116 can act on any appropriate text file type, such as a word file, a PDF file, or a file having another file type.

The text preprocessing system 116 implements a text data exchange protocol 116a. For example, the text preprocessing system 116 can act on text files having any suitable format for EHRs. The text data exchange protocol 116a feeds the raw data 113c to the text preprocessing system 116 and receives the results of text preprocessing, and stores the resultant preprocessed data 113d to the data storage system 112. The text data exchange protocol 116a defines a payload structure (e.g., a structure or format in which raw data is stored within the EHR), and/or metadata associated with the EHR and/or the raw data. The text data exchange protocol 116a provides a way of representing input clinical documents and processed documents output.

In an example, the text data exchange protocol 116a defines, within the pre-processed data 113d, one or more of the following: (i) document name, (ii) document binary content, (iii) document version number, (iv) a date of the patient encounter and/or a data at which the autonomous medical coding system 100 acted on the corresponding EHR, (v) one or more methods used for text extraction from the EHR, (vi) text content derived from the raw data 13c, and/or (vii) the processed text.

In an example, the text preprocessing system 116 implements a text extraction service 116d that extracts text from ingested documents (such as ingested raw data 113c). In an example, the text extraction service 116d uses an optical character recognition (OCR) technique to extract text from the raw data 113c. In another example, the raw data 113c may be in a readable text format, and hence, OCR techniques may not have to be employed.

In an example, the text preprocessing system 116 further implements a de-identification service 116b. The de-identification service 116b identifies and redacts or removes personally identifiable information (PII) from the extracted text. Thus, the preprocessed data 113d stored in the data storage system 112 may not include personally identifiable information of the patient.

In an example, the text preprocessing system 116 further implements a text cleanup service 116c. The text cleanup service 116c performs additional cleanup of the preprocessed data 113d, to remove any unnecessary information and markup text, such as page numbers, barcode values from headers, hospital seals, etc.

Thus, the text preprocessing system 116 receives raw data 113c from the data storage system 112, where the raw data 113c includes EHRs. The text preprocessing system 116 performs text extraction, text de-identification, and/or text cleanup of the raw data 113c, and stores the resultant pre-processed data 113d to the data storage system 112.

The autonomous medical coding system 100 further includes a natural language processing (NLP) system 120. The NLP system 120 is configured to receive the pre-processed data 113d from the data storage system 112, or directly from the text preprocessing system 116. The NLP system 120 processes the pre-processed data 113d, such as performs natural language processing on the pre-processed data 113d, e.g., to find potential points of interest within the pre-processed data 113d.

The NLP system 120 implements a model data exchange protocol 121c, which is a protocol designed to exchange data between the NLP system 120 and a ML model backend service (such as a model backend service 124 described below). The model data exchange protocol 121c facilitates use of any pluggable model backends that work on various coding systems. For example, a first model backend may have a first data exchange protocol, and a second model backend may have a second data exchange protocol, where the first model backend may be used by a first organization in a first region or a first country, and where the second model backend may be used by a second organization in a second region or a second country. The model data exchange protocol 121c facilitates use of any of the first or second model backends with the NLP system 120, in an example.

In an example, the model data exchange protocol 121c defines a way of interaction between the NLP system 120 and the model backends. It defines data exchange of one or more of the following types: (i) target model type, (ii) input text, (iii) raw model output, (iv) predicted codes (e.g., with nomenclature and/or encounter type), and/or (v) output reasoning.

In an example, the NLP system 120 further implements a LLM 121a. The LLM 121a is an ML model that performs text chunking, e.g., to find potential points of interest within the extracted text of the preprocessed data 113d, where the potential points of interest are sections of the text relevant to assignment of medical codes.

The LLM 121a performs the binary classification on an extracted text (such as a chunk of text) of the pre-processed data 113d, e.g., to determine whether the provided text has codable medical information. Thus, a binary classification model is implemented, to determine a codability of the extracted text of the pre-processed data 113d. For example, the codability of the portion of the extracted text is based on whether or an extent to which the portion of the extracted text includes information for assignment of one or more codes. For example, a codability of a portion of the extracted text is positive, if the binary classifier determines that a probability of the extracted text including codable medical data is higher than a threshold value. For example, the NLP system 120 identifies one or more portions of the pre-processed data 113d, which includes information that can be used for (or is pertinent to) medical coding. Merely as an example, in a medial patient record, some sections of the text may be pertinent to medical coding, while some other sections of the text may not be pertinent to medical coding. The NLP system 120, in an example, aims to identify those sections of the text, which are pertinent to medical coding.

In an example, the LLM 121a performs chunking of texts of various sizes and may increase (or decrease) context window (e.g., a window including text that comprises potential points of interests). For example, the chunk of text selected by the LLM 121a may include a sentence, a paragraph, a section, and/or a whole page of the of the preprocessed data 113d.

In an example, the NLP system 120 further implements an input construction service 121b, which constructs an input using extracted text, detected features, and one or more metadata associated with the extracted text. In an example, the input construction service 121b may act on, or otherwise use output of the LLM 121a. The output of the input construction service 121b may be provided to the model backend service 124.

In an example, the autonomous medical coding system 100 further includes the model backend service 124. The model backend service 124 hosts one or more AI and/or ML models for autonomous coding, grouping or bundling of codes, text cleanup, classification, text generation, and/or other tasks. In an example, the model backend service 124 may be implemented in a graphic processing unit (GPU), although other types of processing units (such as central processing unit or CPU) may also be used instead.

The model backend service 124 receives the extracted and processed texts from the NLP system 120 using the model data exchange protocol 121c. In one example, the output of the NLP system 120 can be directly fed to the model backend service 124 from the NLP system 120. In another example, the output of the NLP system 120 may be stored in the data storage system 112 and can be fed to the model backend service 124 from the data storage system 112.

In an example, the model backend service 124 comprises one or more machine learning models, such as one or more coding models 125a and/or one or more grouping models 125b.

The coding models 125a are configured to assign one or more codes to an encounter between a patient and one or more health care professionals. For example, based on the text parsed, chunked, classified as being codable (e.g., codability being positive, as described above), and/or highlighted by the NLP system 120, the coding models 125a assign codes to the encounter results. In an example, coding models 125a for coding and/or evaluation are represented by multi-label classification models, which are based on LLMs pre-trained on large medical corpora and/or generative AI models, pre-trained for general purpose tasks. In an example, more customized models may also be used, wherein a customized model perform feature extraction (e.g., extracts features from given texts parts containing medical coding information, as generated by the NLP system 120) and/or performs reasoning detection (e.g., aims to find within the extracted text reasoning for assigning a medical code, generate code reasoning, and/or provide a reference to particular place of document with information relevant to the code). Training of such coding models 125a will be described below in further detail.

The grouping models 125b may be used for grouping two or more of the medical components of the EHR to form a bundled or group code. For example, bundling or grouping aims to group two or more medical components and assigns a single code to the group. Grouping aims to streamline billing and reimbursement and avoid overpayment or double counting for related services. For example, during a single patient encounter, multiple diagnosis and/or procedures may be performed, where such multiple diagnosis and/or procedures may be grouped or bundled under a single medical code or may be assigned individual medical code.

Merely as an example, if a nosebleed occurs during a nasal endoscopy, the nosebleed may be treated using cauterization. However, a single code may be assigned for the nasal endoscopy, which covers both the nasal endoscopy and the nasal cauterization. In this example, instead of assigning a first code for the nasal endoscopy and a second code for the nasal cauterization, a single code is assigned for nasal endoscopy, as the nasal cauterization may already be covered by nasal endoscopy. However, if another patient needs a nasal cauterization for an unrelated reason (such as nose-bleed due to an injury during a game), the nasal cauterization in this case may be assigned a separate code.

In another example, a surgical procedure may involve incision, the actual procedure, and closure of the incision. However, the incision, the actual procedure, and closure of the incision may be grouped or bundled in a single medical code for the surgical procedure, without assigning separate codes for such individual medical components of the surgical procedure.

In an example, the grouping models 125b may be at least in part or fully integrated with the coding models 125a. In an example, the grouping models 125b include LLM models and/or statistical models. LLM models are used for feature extraction and input documents analysis alongside codes from coding models, to predict grouper codes in the provided nomenclature and/or ontology. In an example, statistical and classification models may be used to generate groupers based on probability of some groupers, given the combination of the input codes.

Merely as an example, the coding models 125a may generate one or more codes for an encounter, and the grouping models 125b may investigate to determine if such one or more codes (or one or more medical components) can be grouped in a single grouper code. In another example, the coding models 125a and the grouping models 125b may work together to assign a grouper code to multiple components of a medical diagnosis or procedure.

In an example, the autonomous medical coding system 100 further includes an encoder system 134. The encoder system 134 is responsible for grouping or encoding all previous system outputs and producing a set of billable codes. The encoder system 134 (also referred to herein as a grouper system) is to work with both inpatient and outpatient encounters in any ontology and/or nomenclature. This is possible due to an encoder data exchange protocol (also referred to as a grouper data exchange protocol). Appropriate model is plugged in on-demand while data exchange is the same. In an example, the encoder system 134 is stateless and it does not know in which ontology it does work. In an example, the encoder system 134 has a set of algorithms to work together to get optimal or near-optimal results possible with emphasis on error minimization.

In an example, the encoder system 134 implements an encoder data exchange protocol 135a. This protocol is designed to handle input and output for various encoders/groupers to make the system versatile. It is a protocol with clear data structure which can use any container for transport. Protocol defines a data frame which contains all required information such as (i) encounter type, (ii) diagnosis codes with nomenclature name and effective date, (iii) procedural codes with nomenclature name and effective date, (iv) country of application, (v) documents, and/or (vi) language. In an example, this protocol is designed to support various inpatient/outpatient (combined with various nomenclature/ontologies) encounter type by keeping data exchange the same for all grouper scenarios. In an example, the grouper/encoder is also stateless. It may not need any state since the communication is done using standard protocol in a standard and defined way.

In an example, the encoder system 134 includes one or more probability-based groupers or encoders 135b, which is based on probability, such as using TF-IDF, PMI, KNN, and other algorithms. Each of these algorithms have been described above herein. In an example, the encoder system 134 works on a voting-based system, where probability scores from multiple encoders are combined to generate a final result, e.g., to increase a confidence of the final result. In an example, the set of various metrics is used for proper grouping to minimize an error chance. Voting system performs gathering multiple metrics from multiple algorithms, and makes a decision based on that.

In an example, the encoder system 134 includes one or more LLM-based groupers or encoders 135c. A set of groupers or encoders based on LLMs, such as Bidirectional Encoder Representations from Transformers® (BERT), Generative Pre-trained Transformer® (GPT), and/or other LLMs may be used. In an example, LLM-based groupers or encoders may be used in combination with one or more probability-based groupers. In difference from probability-based encoders, LLM-based encoders take as input not only coding results, but also context (such as clinical documents and other input in natural language, e.g., output of the NLP system 120).

In an example, the autonomous medical coding system 100 further includes a validation system 132. The validation system 132 performs various methods of analysis to find potential erroneous coding results, e.g., based on various probabilistic and statistical metrics. The combination of all metrics gives a final probability score of these coding results being correct (or incorrect).

In an example, the validation system 132 implements a plurality of statistical and/or probabilistic models, and/or language models (such as LLMs) to predict a probability of the assigned codes being correct. The validation system 132 may process codes generated by the model backend service 124 and/or billable codes generated by the encoder system 134. FIG. 1A illustrates some examples of such statistical and/or probabilistic models 133a, 133b, 133c, although a number and/or a nature of the statistical and/or probabilistic models may vary from one implementation to the next.

FIG. 1B illustrates a validation operation on assigned codes (or billable codes) generated by the model backend service 124 and/or the encoder system 134 of the autonomous medical coding system 100 of FIG. 1A. While FIG. 1A illustrates some specific examples of the statistical and/or probabilistic models 133a, 133b, 133c, different types and/or number of such statistical and/or probabilistic models may be used.

Referring to FIGS. 1A and 1B, in an example, the validation system 132 implements a probability mutual information (PMI) model 133a. The PMI model 133a is an algorithm that predicts a conditional probability of the event of code A, given a code B event occurred. In one example, the grouping models 125b may group two codes A and B into a single code C. In another example, the coding models 125a assigns two codes A and B for a single patient encounter. The PMI model 133a provides a conditional probability of the event of code A, given a code B event occurred. Thus, if this probability is less than a threshold value, then the codes assigned by the model backend service 124 may possibly be erroneous, or at least may require a human coder review. In an example, the PMI model 133a generates a probability of two or more codes to be predicted together for a single given patient encounter, and a probability of such two or more codes not to be predicted together for a single given patient encounter.

Thus, as illustrated in FIG. 1B, the PMI model 133a receives two or more codes 180 assigned (e.g., by the model backend service 124 or the encoder system 134) to the health record of the encounter being validated. The PMI model 133a outputs a probability score 185a, which may be a conditional probability of a first one of the two or more codes 180 being assigned to a single given encounter, given that remaining ones of the two or more codes 180 is assigned to the single given encounter. Note that the health record and/or the extracted text, based on which the codes 180 are assigned, are not used by the PMI model 133a to generate the probability score 185a (e.g., as the model 133a relies on the codes 180, and not on documents from which the codes 180 are derived, to generate the probability score).

In an example, the validation system 132 further implements a TF-IDF (term frequency- inverse document frequency) based model 133b. The TF-IDF is the product of two statistical term, term frequency and inverse document frequency. There are various ways for determining the exact values of both these statistical terms. The TF-IDF is a measure of importance of a word (or in this case, a code) to a document in a collection or corpus, adjusted for the fact that some words appear more frequently in general. An adapted TF-IDF metric shows the probability and uniqueness of code combinations. This metric measures probability and uniqueness of each code and code combination to give another probability score. Thus, when the grouper models 125b groups multiple codes (or components of a medical procedure) in a single code, the TF-IDF metric measures probability and uniqueness of each such individua code and the code combination.

Thus, as illustrated in FIG. 1B, the TF-IDF model 133b receives the two or more codes 180 assigned to the health record of the encounter being validated. The model 133b outputs a probability score 185b, which may be indicative of a correctness of a combination of the two or more codes 180. In an example, the health record and/or the extracted text, based on which the codes 180 are assigned, are not used by the model 133b to generate the probability score 185b (e.g., as the model 133b relies on the codes 180, and not on documents from which the codes 180 are derived, to generate the probability score).

In an example, the validation system 132 further implements a KNN (k-nearest neighbors) model 133c. The KNN model 133c generates a statistical metric indicative of how often the assigned codes appear together. Thus, for the above-described example where the model backend service 124 has assigned codes A and B for a single patient encounter (either individually as codes A and B, or as a grouped or bundled code C), the KNN model 133c generates a statistical metric or a probability of codes A and B appearing together for a single patient encounter. In another example, the KNN model 133c generates a statistical metric or a probability of codes A and B not appearing together for a single patient encounter. The KNN model 133c uses a clustering approach based on KNN algorithms. The KNN model 133c aims to detect codes that usually do not appear together. The KNN model 133c provides an opposite information to the PMI model 133a and are used to mitigate Type II errors.

Thus, as illustrated in FIG. 1B, the KNN model 133c receives the two or more codes 180 assigned to the health record of the encounter being validated. The model 133c outputs a probability score 185c, which may be indicative of a correctness of a combination of the two or more codes 180. In an example, the health record and/or the extracted text, based on which the codes 180 are assigned, are not used by the model 133c to generate the probability score 185b (e.g., as the model 133a relies on the codes 180, and not on documents from which the codes 180 are derived, to generate the probability score).

In an example, the validation system 132 further implements a language model 133b (such as a LLM). The language model 133d receives the two or more codes 180 assigned to the health record of the encounter being validated, as well as receives reference documents 183 (such as raw data 113c, pre-processed data 113d, extracted text, and/or data generated by the NLP system 120 and used by the model backend service 124 to generate the coding results). Based on the reference documents 183, the language model 133d generates a probability score 185d of the assigned codes 180 being correct. In an example, the language model 133d uses a classification approach and/or a generative approach to generate the probability score 185d.

In an example, the validation system 182 further includes a scoring service 186 that receives the probability scores 185a, . . . , 185d, and generates a final probability score 187 indicative of a probability of the assigned codes 180 being correct. In an example, the scoring service 186 relies on a voting-based system, where probability scores from multiple models are combined to generate a final result. The scoring service 186 may average (such as a weighted average) the probability scores 185a, . . . . , 185d (or perform another operation of the probability scores 185a, . . . , 185d), to arrive at the final probability score 187. The final probability score 187 being higher than a threshold score implies that the assigned codes 180 are correct.

For example, if the final probability score 187 is higher than a high threshold, the codes 180 are assumed to be correct and approved without human intervention or verification. If the final probability score 187 is between the high threshold and a low threshold, validity of the codes 180 are assumed to be questionable, and the codes 180 are transmitted to a human coder for manual verification. If the final probability score 187 is less than the low threshold, the codes 180 are assumed to be incorrect, and the reference documents 183 (such as raw data 113c, pre-processed data 113d, and/or data generated by the NLP system 120 and used by the model backend service 124 to generate the coding results) are provided to a human coder for re-coding. In an example, codes 180 that are verified by human coders or recoded by human coders may be used to train one or more machine learning models of the autonomous medical coding system 100, as described below in further detail.

Referring again to FIG. 1A, in an example, the autonomous medical coding system 100 further includes a user interface (UI) system 136 that includes a plurality of UIs. Appropriate types of UIs may be used, such as a display screen, or another type of UI. One or more users interact with the autonomous medical coding system 100 through the UIs.

The UI system 136 includes a results review and correction UI 137a, which displays a final stage of medical coding, such as displays the assigned codes 180 and/or the final probability score 187. When codes are assigned, as user (such as a human coder) of the autonomous medical coding system 100 may approve or reject the coding results. In another example, approval could happen automatically (e.g., by the system 100, without a user reviewing the coding results), if the validation system 132 approves the coding results. As and when the ML models of the system 100 are more trained, the system 100 may gradually transition from the manual review of the coding results to the auto validation of the coding results by the validation system 132.

The UI system 136 includes a browse cases/documents UI 137b. In an example, this UI 137b enables users to browse cases/coding results and other statistical information. The UI system 136 includes a grouper/encoder UI 137c, which displays results of the encoder system 134.

In an example, the autonomous medical coding system 100 further includes a data augmentation system 114, which augments the coding results, the raw data 113c, the preprocessed data 113d, the training data 113b, and/or other data generated by the system 100. For example, after such augmentation, the training data 113b is used to train the models 125a, 125b.

For example, the data augmentation system 114 includes a results statistical analysis service 115b. This service performs statistical analysis of the coding results. This system performs analysis of the codes that are often clubbed together for a single encounter, and/or identifies encounters and/or codes for which the final probability score 187 is less than a threshold value (e.g., identifies scenarios where failures or errors occurs). This information may be used to augment data to cover those use-cases to improve the system.

For example, the data augmentation system 114 also includes a text augmentation system 115a. This system performs text augmentation and generates new training examples based on statistics from the results statistical analysis service. Data augmentation is useful in a scenario where a ML model of the backend service 124 (such as the coding models 125a and/or the grouping models 125b) has to be re-trained, e.g., to address some errors, but there may not be sufficient data for such retraining. Data augmentation allows to generate new data, based on relatively small set of existing training data, thereby increasing overall amount of training data examples.

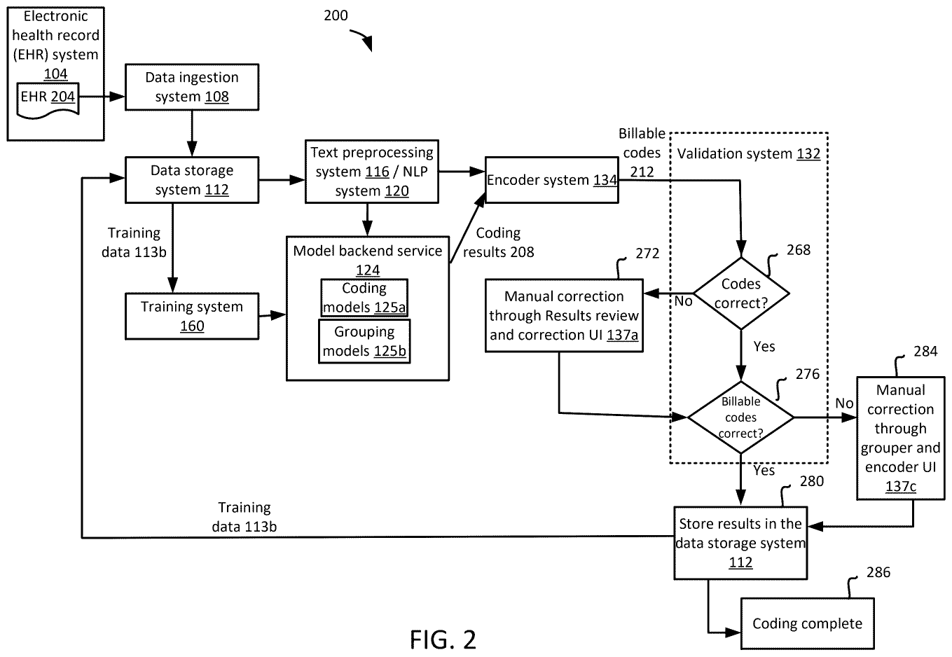

In an example, the autonomous medical coding system 100 further includes a model training system 160, which includes a model training service 161b. The model training service 161b may be used for model training (such as continuous model training) of the coding models 125a and/or the grouping models 125b. The models 125a, 125b are trained using labelled training data received from sources external to the system 100 and/or using training data generated by the system 100 (generation of training data is described below and also discussed with respect to FIG. 2). In an example, the models 125a, 125b are trained on commercially available cloud services, such as Oracle Cloud Service® (OCI®), HuggingFace®, Amazon Web Services® (AWS®), and/or the like.

The model training system 160 also includes a dataset generation service 161a, which generates training data 113b usable to train the models 125a, 125b. The dataset generation service 161a generates the training dataset based on model performance, samples it adequately, and prepares for training. Depending on system results and statistical analysis, the dataset generation service 161a takes data from database and creates balanced dataset. For example, various statistical and probabilistic metrics are used alongside pre-augmented text from the text augmentation system 115a for training purposes.

FIG. 2 illustrates a flow diagram 200 illustrating operations of the autonomous medical coding system 100 of FIG. 1A. The data ingestion system 108 receives an EHR 204 from the EHR system 104 (e.g., through the data pull interface 109 or the data push interface 110), as also described above. The data ingestion system 108 stores the EHR 204 in the data storage system 112 (e.g., as raw data 113c).

The text preprocessing system 116 and/or the NLP system 120 processes the EHR 204. For example, the text preprocessing system 116 processes the EHR 204, and generates and stores preprocessed data 113d to the data storage system 112. The NLP system 120 processes the preprocessed data 113d, and output of the NLP system 120 is stored in the data storage system 112 and/or is provided to the model backend service 124.

The model backend service 124 (such as the coding models 125a and/or the grouping models 125b) processes the output of the NLP system 120, to generate the coding results 208, as also described above.

The encoder system 134 receives the coding results 208. In an example, the encoder system 134 also receives the output of the NLP system 120. The encoder system 134 generates the final billable codes 212. For example, the probability-based encoders 135b of the encoder system 134 processes the coding results 208, to generate probability values. In contrast, the LLM based encoder 135c processes both the coding results 208 and the output of the NLP system 120, to generate probability values.

The encoder system 134 generates the billable codes 212. In an example, the coding results 208 and the billable codes 212 are the same. In another example, the coding results 208 and the billable codes 212 may be different. For example, the encoder system 134 may assign modifiers to the coding results 208, to generate the billable codes 212. In another example, the encoder system 134 may further group or bundle one or more codes of the coding results, to generate the billable codes 212. In yet another example, the encoder system 134 assigns probability values to the groupings of the codes, which may also be used by the validation system 132 for validation purposes. In an example, the encoder system 134 facilitates in assigning proper codes by providing one or more toolkits, such as semantic search, narrowing questions, etc. The encoder system 134 may have a dual role. For machine-to-machine interaction (e.g., server-server interaction) the encoder system 134 groups (e.g., compiles) one or more or more medical and/or procedural codes together, thereby providing the final output. For human-to-machine interaction, for example through an UI, the encoder system 134 facilitates in assigning the billable codes 212 through an UI, e.g., by asking questions to the human user (such as a coder or a biller) and provides a convenient interface for generating the billable codes 212.

The billable codes 212 are received by the validation system 132 from the encoder system 134. The validation system 132 is illustrated in FIG. 2 using a dotted box, with operations performed by the validation system 132 depicted within this dotted box.

The validation system 132 at 268 checks to determine if the assigned codes in the coding results 208 are correct (e.g., assigns the final probability score 187, see FIG. 1B). If the codes are incorrect at 268 (e.g., “No” at 268 when, for example, the final probability score 187 is lower than a threshold), at 272, the assigned codes in the coding results 208 are displayed in a results review and correction UI 137a, and a medical coder manually checks the assigned codes, and performs correction if needed. Then the flow proceeds from 272 to 276.

On the other hand, if the codes are correct at 268 (e.g., “Yes” at 268, when, for example, the final probability score 187 is higher than the threshold), flow proceeds from 268 to 276.

At 276, the validation system 132 checks to determine if the final billable codes 212 are correct (e.g., whether a probability of the billable codes 212 being correct is higher than a threshold value, or whether a probability of the billable codes 212 being incorrect is lower than another threshold value). Note that in an example, the process flow of FIG. 1B applies to the codes generated by the model backend service 124 and/or the billable codes 212 generated by the encoder system 134. If the billable codes 212 are incorrect at 276 (e.g., “No” at 276 ), the flow proceeds from 276 to 284. At 284, the billable codes 212 are displayed in the grouper and encoder UI 137c, and a medical coder manually checks the billable codes 212, or performs correction, if needed. Then the flow proceeds from 284 to 280.

On the other hand, if the billable codes 212 are correct at 276 (e.g., “Yes” at 276), flow proceeds from 276 to 280. At 280, the final billable codes (either without correction, or with correction if needed) and other relevant data (such as extracted text by the NLP system, the raw data, etc.) are stored in the data storage system 112, and may be used as training data. Note that the data augmentation system 114 may augment the training data 113b subsequent to the training data 113b being stored in the data storage system 112. At 286, the final billable codes (either without correction, or with correction if needed) are output and the coding is complete for the EHR 204.

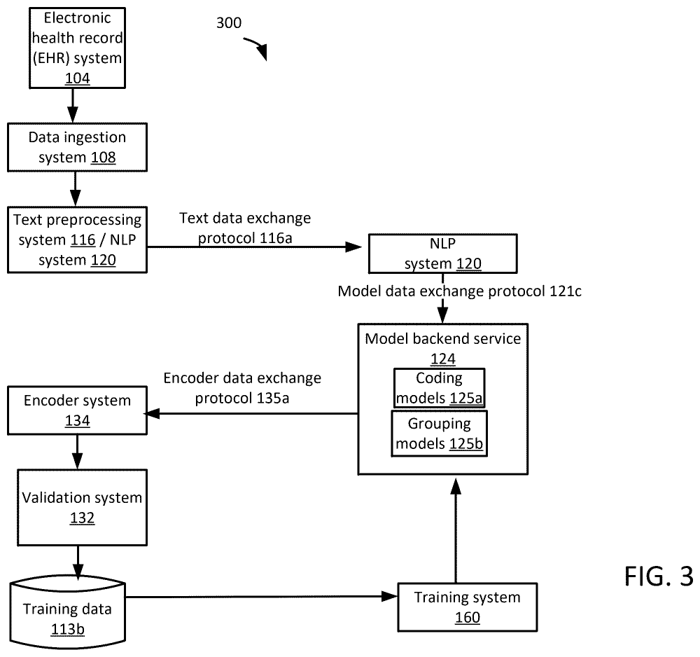

FIG. 3 illustrates a flow diagram 300 illustrating data flow within the autonomous medical coding system 100 of FIG. 1A. FIG. 3 will be self-evident, based on the above description with respect to FIGS. 1A, 1B, and 2.

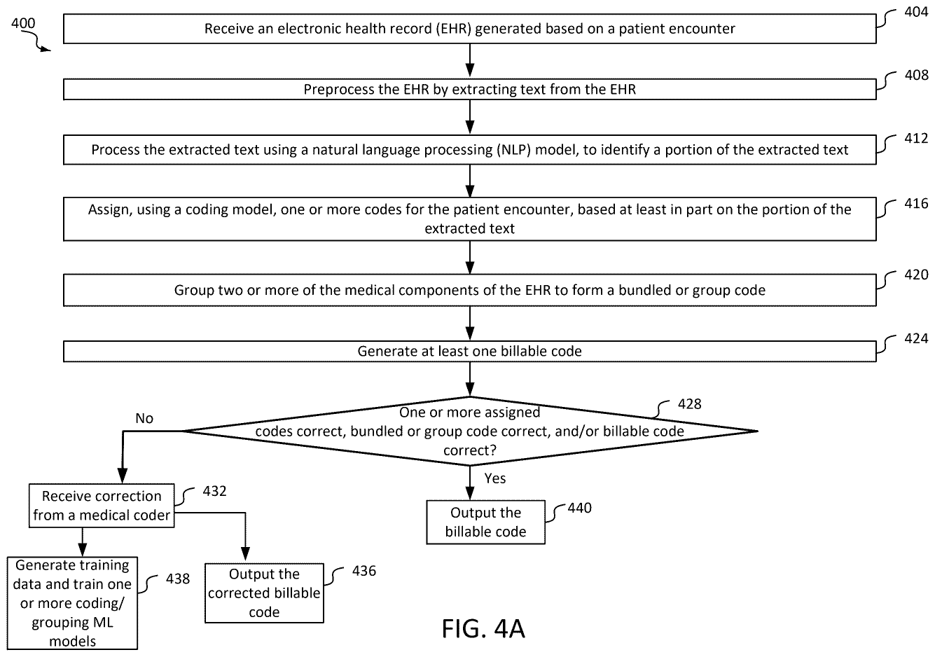

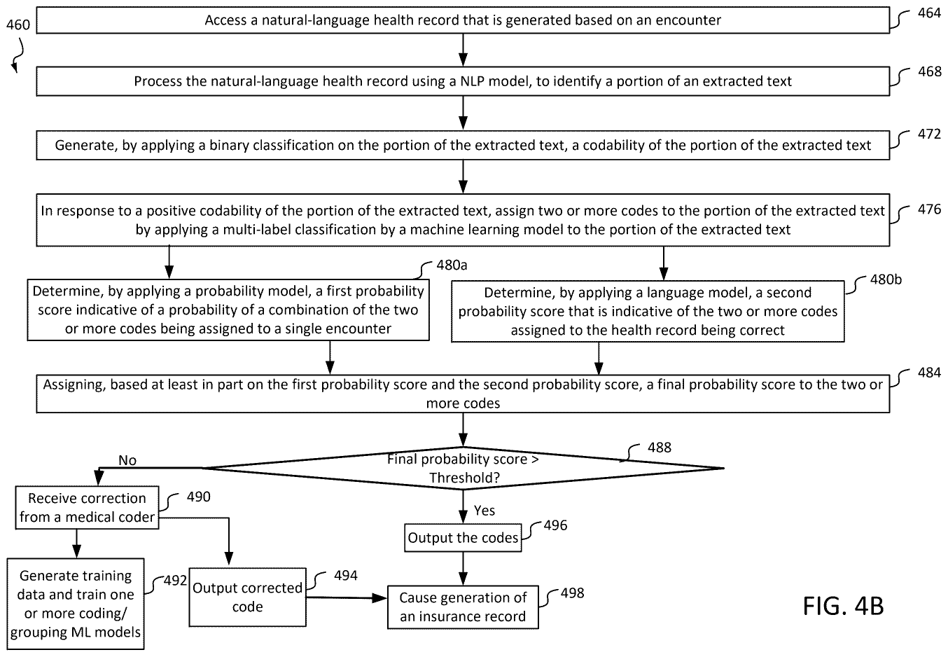

FIG. 4A illustrates a method 400 for autonomously assigning billable codes to a patient encounter. The method 400 can be implemented within the system 100 of FIG. 1A, also described above with respect to FIGS. 1B, 2, and 3.

At 404, an electronic health record (EHR) generated based on a patient encounter is received (e.g., by a data ingestion system, either via push or pull, as described above). At 408, the EHR is preprocessed, by extracting text from the EHR (e.g., by the text preprocessing system 116). At 412, the extracted text is processed using a natural language processing (NLP) model (e.g., by the NLP system 120), to identify a portion of the extracted text that is relevant to assigning codes. At 416, using a coding model, one or more codes are assigned for the patient encounter, based at least in part on the portion of the extracted text (e.g., by the coding models 125a).

At 420, optionally two or more of the medical components of the EHR may be grouped to form a bundled or group code (e.g., by the grouping models 125b). At 424, at least one billable code is generated (e.g., by the encoder system 134). At 428, a determination is made as to whether the one or more assigned codes, the bundled or group code, and/or billable code are correct (e.g., by the validation system 132). If “Yes” at 428, at 440, the billable code is output and the autonomous coding of the EHR is complete. If “No” at 428, at 432, one or more corrections of the one or more assigned codes, the bundled or group code, and/or billable code are received, where the corrections are performed manually by a medical coder. At 436, the corrected billable code is output and the autonomous coding of the EHR is complete.

Also, at 438, the corrected code, along with the extracted text and/or the EHR are used to generate training data, and the training data is used to train one or more coding/grouping ML models (e.g., by the training system).

FIG. 4B illustrates another method 460 for autonomously assigning and validating billable codes to a patient encounter. The method 460 can be implemented within the system 100 of FIG. 1A, also described above with respect to FIGS. 1B, 2, and 3.

At 464, a natural-language health record, which is generated based on a patient encounter, is accessed, e.g., by the autonomous medical coding system 100. For example, the health record may be pulled by the data ingestion system 108 from the EHR system 104, or pushed to the data ingestion system 108 from the EHR system 104. The health record may be stored in a storage repository within the data storage system 112.

At 468, the natural-language health record may be processed using an NLP model, to identify a portion of an extracted text from the natural-language health record. For example, the text preprocessing system 116 preprocesses the health record and extracts text from the health record. The NLP system 120 identifies a portion of the extracted text.

At 472, by applying a binary classification on the portion of the extracted text, a codability of the portion of the extracted text is generated. For example, the NLP system performs binary classification on the portion of the extracted text, e.g., to determine whether the provided text has codable medical information. The codability of the portion of the extracted text is based on whether or an extent to which the portion of the extracted text includes information for assignment of one or more codes.

At 476, in response to a positive codability of the portion of the extracted text (e.g., when codability is greater than a threshold), two or more codes are assigned to the portion of the extracted text by applying a multi-label classification by a machine learning model to the portion of the extracted text. For example, the coding models 125a, the grouping models 125b, and/or the encoder system 134 assign the two or more codes. In an example, when the encoder system 134 assigns the two or more codes, the codes are billable codes. In an example, subsequent processes 480a, 480b, . . . . , 496 are applicable to codes generated by the coding models 125a and/or the grouping models 125b, as well as billable codes generated by the encoder system 134.

The method 460 proceeds from 476 to 480a and 480b. Here in 480a, a single probability model is assumed. However, the method 460 can be extended to scenarios where there may be more than one such probability model and/or statistical model, as described below with respect to FIG. 1B.

At 480a, by applying a probability or statistical model, a first probability score is determined, where the first probability score is indicative of a probability of a combination of the two or more codes being assigned to a single encounter. The first probability score may be any of the scores 185a, 185b, or 185c (or a combination of such scores) described above.

At 480b, by applying a language model, a second probability score is determined, where the second probability score is indicative of the two or more codes assigned to the health record being correct. The second probability score may be the score 185d described above.

The method 640 proceeds from 480a and 480b to 484. At 484, based at least in part on the first probability score and the second probability score, a final probability score is assigned to the two or more codes. The final probability score has been described above with respect to FIG. 1B.

At 488, a determination is made as to whether the final probability score is higher than a threshold. If “Yes” at 488, at 496, the two or more codes are output. Note that if the two or more codes are generated by the coding models 125a or the grouping models 125b, one or more billable codes may be generated after 496 from to the two or more codes of FIG. 4B.

If “No” at 488, at 490, one or more corrections of the two or more assigned codes are received, where the corrections are performed manually by a medical coder. At 494, the corrected two or more codes are output.

Also, at 492, the corrected codes, along with the extracted text and/or the health record are used to generate training data, and the training data is used to train one or more coding and/or grouping ML models (e.g., by the training system).

The method 460 proceeds from 496 and 494 to 498. At 498, generation of an insurance record is caused. The insurance record may be a reimbursement request from a health insurance carrier, which may include the codes (or billable codes) of 494 and 496.

The autonomous medical coding system 100 comprises an end-to-end artificial intelligence/ML based system for multiple healthcare specialties and healthcare domains. In an example, the architecture and building blocks of the autonomous medical coding system 100 are designed to fit the healthcare regulations of different countries or regions, by introducing universal data exchange protocols. For example, the same autonomous medical coding system 100 may be used for different countries and/or nomenclature additions. Since various data exchange protocols described above are used, it may be possible to: (i) deploy infrastructure, (ii) have a valid consumer within the system (model can consume input defined by nomenclature, country, region, etc.). As described above, the autonomous medical coding system 100 provides interfaces for either pull or push data ingestion. Accordingly, the autonomous medical coding system 100 can be integrated into any EHR system providing EHRs, without breaking or replacing the current component, or adding additional and parallel execution lines.

In an example, the system operates on top of multiple data interfaces and/or data exchange protocols described above. An example of such a data interface and/or data exchange protocol includes the text data exchange protocol 116a, which allows exchange of medical documents in one of multiple file formats.

Another example of such a data interface and/or data exchange protocol includes the model data exchange protocol 121c, which may be used to exchange information related to medical coding between various components of the autonomous medical coding system 100. This makes the autonomous medical coding system 100 independent of a specific country or a specific vendor. Thus, the same autonomous medical coding system 100 may be used in a new region or country (e.g., having corresponding coding system) or with components from a new vendor. In such a scenario, an appropriate model backend service 124 (e.g., which supports the desired coding system and/or ontology specific to the country and its coding regulation) may be plugged in the autonomous medical coding system 100.

Another example of such a data interface and/or data exchange protocol includes the encoder data exchange protocol 135a, which dictates a manner in which data is exchanged between the encoder system 134 and other components of the autonomous medical coding system 100.

In an example, the autonomous medical coding system 100 performs an analysis of individual health records and removes PII as well as non-coding related data (such as hospital logo, page number, etc.), leaving text that includes codable information. In an example, the autonomous medical coding system 100 performs error analysis, to identify possibly erroneous codes assigned by the autonomous medical coding system 100, e.g., by implementing a multi-level validation system 132. This validation system 132 is based on set of customized models based on various LLMs architectures, such as Bidirectional Encoder Representations from Transformers® (BERT), Generative Pre-trained Transformer® (GPT), and/or other transformer-based models, including models of multi-label classification with adaptive threshold, as well as probabilistic and statistical models, e.g., as described above with respect to FIG. 1B. In an example, the autonomous medical coding system 100 uses positive results (e.g., correct coding outcomes) and negative results (marked as wrong by the validation system 132 and/or by human coders) to generate proper training data and/or training strategy to address such errors.

In an example, the autonomous medical coding system 100 improves the time spent on each encounter to get it coded. Thus, the autonomous medical coding system 100 does not require human attention to code an encounter. Instead, the autonomous medical coding system 100 requests the coder's attention to review results, e.g., especially for scenarios in which the final probability score 187 is less than a high threshold value, as described above with respect to FIG. 1B.

### Computer System Architecture

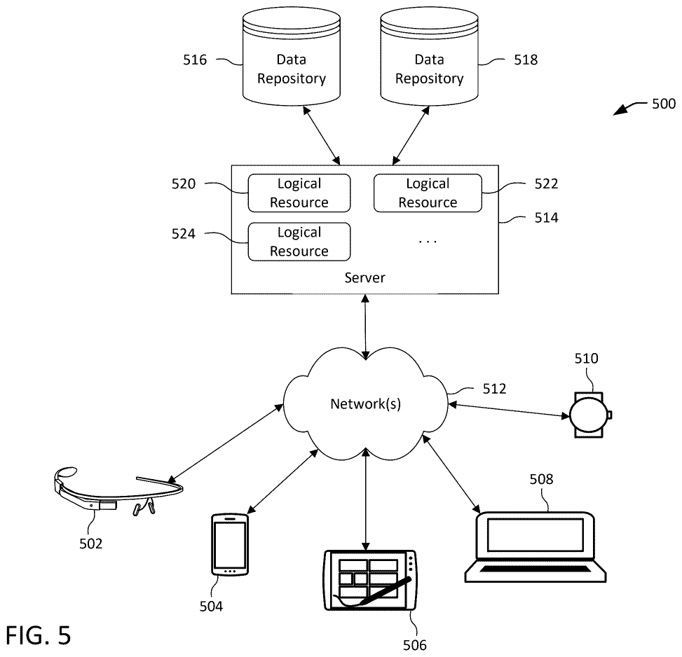

FIG. 5 depicts a simplified diagram of a distributed system 500 for implementing an embodiment. In the illustrated embodiment, distributed system 500 includes one or more client computing devices 502, 504, 506, 508, and/or 510 coupled to a server 514 via one or more communication networks 512. Clients computing devices 502, 504, 506, 508, and/or 510 may be configured to execute one or more applications.

In various aspects, server 514 may be adapted to run one or more services or software applications that enable techniques for autonomous medical coding.

In certain aspects, server 514 may also provide other services or software applications that can include non-virtual and virtual environments. In some aspects, these services may be offered as web-based or cloud services, such as under a Software as a Service (SaaS) model to the users of client computing devices 502, 504, 506, 508, and/or 510. Users operating client computing devices 502, 504, 506, 508, and/or 510 may in turn utilize one or more client applications to interact with server 514 to utilize the services provided by these components.

In the configuration depicted in FIG. 5, server 514 may include one or more components 520, 522 and 524 that implement the functions performed by server 514. These components may include software components that may be executed by one or more processors, hardware components, or combinations thereof. It should be appreciated that different system configurations are possible, which may be different from distributed system 500. The embodiment shown in FIG. 5 is thus one example of a distributed system for implementing an embodiment system and is not intended to be limiting.

Users may use client computing devices 502, 504, 506, 508, and/or 510 for techniques for autonomous medical coding in accordance with the teachings of this disclosure. A client device may provide an interface that enables a user of the client device to interact with the client device. The client device may also output information to the user via this interface. Although FIG. 5 depicts only five client computing devices, any number of client computing devices may be supported.

The client devices may include various types of computing systems such as smart phones or other portable handheld devices, general purpose computers such as personal computers and laptops, workstation computers, personal assistant devices, smart watches, smart glasses, or other wearable devices, equipment firmware, gaming systems, thin clients, various messaging devices, sensors or other sensing devices, and the like. These computing devices may run various types and versions of software applications and operating systems (e.g., Microsoft Windows®, Apple Macintosh®, UNIX® or UNIX-like operating systems, Linux® or Linux-like operating systems such as Oracle® Linux and Google Chrome® OS) including various mobile operating systems (e.g., Microsoft Windows Mobile®, iOS®, Windows Phone®, Android®, HarmonyOS®, Tizen®, KaiOS®, Sailfish® OS, Ubuntu® Touch, CalyxOS®). Portable handheld devices may include cellular phones, smartphones, (e.g., an iPhone®), tablets (e.g., iPad®), and the like. Virtual personal assistants such as Amazon® Alexa®, Google® Assistant, Microsoft® Cortana®, Apple® Siri®, and others may be implemented on devices with a microphone and/or camera to receive user or environmental inputs, as well as a speaker and/or display to respond to the inputs. Wearable devices may include Apple® Watch, Samsung Galaxy® Watch, Meta Quest®, Ray-Ban® Meta® smart glasses, Snap® Spectacles, and other devices. Gaming systems may include various handheld gaming devices, Internet-enabled gaming devices (e.g., a Microsoft Xbox® gaming console with or without a Kinect® gesture input device, Sony PlayStation® system, Nintendo Switch®, and other devices), and the like. The client devices may be capable of executing various different applications such as various Internet-related apps, communication applications (e.g., e-mail applications, short message service (SMS) applications) and may use various communication protocols.

Network(s) 512 may be any type of network familiar to those skilled in the art that can support data communications using any of a variety of available protocols, including without limitation TCP/IP (transmission control protocol/Internet protocol), SNA (systems network architecture), IPX (Internet packet exchange), AppleTalk®, and the like. Merely by way of example, network(s) 512 can be a local area network (LAN), networks based on Ethernet, Token-Ring, a wide-area network (WAN), the Internet, a virtual network, a virtual private network (VPN), an intranet, an extranet, a public switched telephone network (PSTN), an infra-red network, a wireless network (e.g., a network operating under any of the Institute of Electrical and Electronics (IEEE) 1002.11 suite of protocols, Bluetooth®, and/or any other wireless protocol), and/or any combination of these and/or other networks.

Server 514 may be composed of one or more general purpose computers, specialized server computers (including, by way of example, PC (personal computer) servers, UNIX® servers, LINIX® servers, mid-range servers, mainframe computers, rack-mounted servers, etc.), server farms, server clusters, a Real Application Cluster (RAC), database servers, or any other appropriate arrangement and/or combination. Server 514 can include one or more virtual machines running virtual operating systems, or other computing architectures involving virtualization such as one or more flexible pools of logical storage devices that can be virtualized to maintain virtual storage devices for the server. In various aspects, server 514 may be adapted to run one or more services or software applications that provide the functionality described in the foregoing disclosure.

The computing systems in server 514 may run one or more operating systems including any of those discussed above, as well as any commercially available server operating system. Server 514 may also run any of a variety of additional server applications and/or mid-tier applications, including HTTP (hypertext transport protocol) servers, FTP (file transfer protocol) servers, CGI (common gateway interface) servers, JAVA® servers, database servers, and the like. Exemplary database servers include without limitation those commercially available from Oracle®, Microsoft®, SAP®, Amazon®, Sybase®, IBM® (International Business Machines), and the like.

In some implementations, server 514 may include one or more applications to analyze and consolidate data feeds and/or event updates received from users of client computing devices 502, 504, 506, 508, and/or 510. As an example, data feeds and/or event updates may include, but are not limited to, blog feeds, Threads® feeds, Twitter® feeds, Facebook® updates or real-time updates received from one or more third party information sources and continuous data streams, which may include real-time events related to sensor data applications, financial tickers, network performance measuring tools (e.g., network monitoring and traffic management applications), clickstream analysis tools, automobile traffic monitoring, and the like. Server 514 may also include one or more applications to display the data feeds and/or real-time events via one or more display devices of client computing devices 502, 504, 506, 508, and/or 510.

Distributed system 500 may also include one or more data repositories 516, 518. These data repositories may be used to store data and other information in certain aspects. For example, one or more of the data repositories 516, 518 may be used to store information for techniques for autonomous medical coding. Data repositories 516, 518 may reside in a variety of locations. For example, a data repository used by server 514 may be local to server 514 or may be remote from server 514 and in communication with server 514 via a network-based or dedicated connection. Data repositories 516, 518 may be of different types. In certain aspects, a data repository used by server 514 may be a database, for example, a relational database, a container database, an Exadata® storage device, or other data storage and retrieval tool such as databases provided by Oracle Corporation® and other vendors. One or more of these databases may be adapted to enable storage, update, and retrieval of data to and from the database in response to structured query language (SQL)-formatted commands.

In certain aspects, one or more of data repositories 516, 518 may also be used by applications to store application data. The data repositories used by applications may be of different types such as, for example, a key-value store repository, an object store repository, or a general storage repository supported by a file system.

In one embodiment, server 514 is part of a cloud-based system environment in which various services may be offered as cloud services, for a single tenant or for multiple tenants where data, requests, and other information specific to the tenant are kept private from each tenant. In the cloud-based system environment, multiple servers may communicate with each other to perform the work requested by client devices from the same or multiple tenants. The servers communicate on a cloud-side network that is not accessible to the client devices in order to perform the requested services and keep tenant data confidential from other tenants.

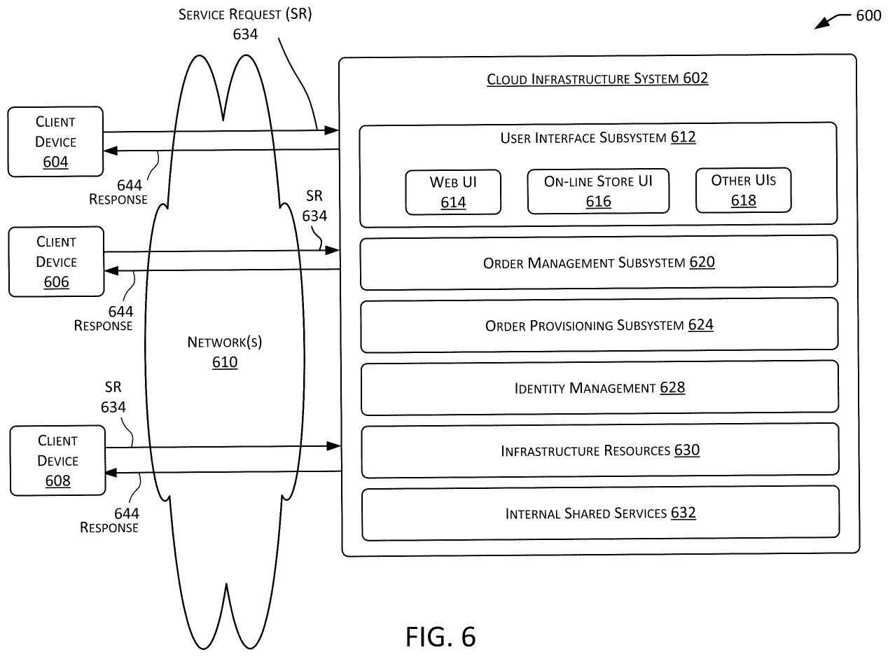

FIG. 6 is a simplified block diagram of a cloud-based system environment in which autonomous medical coding is performed, in accordance with certain aspects. In the embodiment depicted in FIG. 6, cloud infrastructure system 602 may provide one or more cloud services that may be requested by users using one or more client computing devices 604, 606, and 608. Cloud infrastructure system 602 may comprise one or more computers and/or servers that may include those described above for server 514. The computers in cloud infrastructure system 602 may be organized as general purpose computers, specialized server computers, server farms, server clusters, or any other appropriate arrangement and/or combination.

Network(s) 610 may facilitate communication and exchange of data between clients 604, 606, and 608 and cloud infrastructure system 602. Network(s) 610 may include one or more networks. The networks may be of the same or different types. Network(s) 610 may support one or more communication protocols, including wired and/or wireless protocols, for facilitating the communications.

The embodiment depicted in FIG. 6 is only one example of a cloud infrastructure system and is not intended to be limiting. It should be appreciated that, in some other aspects, cloud infrastructure system 602 may have more or fewer components than those depicted in FIG. 6, may combine two or more components, or may have a different configuration or arrangement of components. For example, although FIG. 6 depicts three client computing devices, any number of client computing devices may be supported in alternative aspects.

The term cloud service is generally used to refer to a service that is made available to users on demand and via a communication network such as the Internet by systems (e.g., cloud infrastructure system 602) of a service provider. Typically, in a public cloud environment, servers and systems that make up the cloud service provider's system are different from the cloud customer's (“tenant's”) own on-premise servers and systems. The cloud service provider's systems are managed by the cloud service provider. Tenants can thus avail themselves of cloud services provided by a cloud service provider without having to purchase separate licenses, support, or hardware and software resources for the services. For example, a cloud service provider's system may host an application, and a user may, via a network 610 (e.g., the Internet), on demand, order and use the application without the user having to buy infrastructure resources for executing the application. Cloud services are designed to provide easy, scalable access to applications, resources, and services. Several providers offer cloud services. For example, several cloud services are offered by Oracle Corporation®, such as database services, middleware services, application services, and others.

In certain aspects, cloud infrastructure system 602 may provide one or more cloud services using different models such as under a Software as a Service (SaaS) model, a Platform as a Service (PaaS) model, an Infrastructure as a Service (IaaS) model, a Data as a Service (DaaS) model, and others, including hybrid service models. Cloud infrastructure system 602 may include a suite of databases, middleware, applications, and/or other resources that enable provision of the various cloud services.

A SaaS model enables an application or software to be delivered to a tenant's client device over a communication network like the Internet, as a service, without the tenant having to buy the hardware or software for the underlying application. For example, a SaaS model may be used to provide tenants access to on-demand applications that are hosted by cloud infrastructure system 602. Examples of SaaS services provided by Oracle Corporation® include, without limitation, various services for human resources/capital management, client relationship management (CRM), enterprise resource planning (ERP), supply chain management (SCM), enterprise performance management (EPM), analytics services, social applications, and others.

An IaaS model is generally used to provide infrastructure resources (e.g., servers, storage, hardware, and networking resources) to a tenant as a cloud service to provide elastic compute and storage capabilities. Various IaaS services are provided by Oracle Corporation®.

A PaaS model is generally used to provide, as a service, platform and environment resources that enable tenants to develop, run, and manage applications and services without the tenant having to procure, build, or maintain such resources. Examples of PaaS services provided by Oracle Corporation® include, without limitation, Oracle Database Cloud Service (DBCS), Oracle Java Cloud Service (JCS), data management cloud service, various application development solutions services, and others.

A DaaS model is generally used to provide data as a service. Datasets may searched, combined, summarized, and downloaded or placed into use between applications. For example, user profile data may be updated by one application and provided to another application. As another example, summaries of user profile information generated based on a dataset may be used to enrich another dataset.

Cloud services are generally provided on an on-demand self-service basis, subscription-based, elastically scalable, reliable, highly available, and secure manner. For example, a tenant, via a subscription order, may order one or more services provided by cloud infrastructure system 602. Cloud infrastructure system 602 then performs processing to provide the services requested in the tenant's subscription order. Cloud infrastructure system 602 may be configured to provide one or even multiple cloud services.

Cloud infrastructure system 602 may provide the cloud services via different deployment models. In a public cloud model, cloud infrastructure system 602 may be owned by a third party cloud services provider and the cloud services are offered to any general public tenant, where the tenant can be an individual or an enterprise. In certain other aspects, under a private cloud model, cloud infrastructure system 602 may be operated within an organization (e.g., within an enterprise organization) and services provided to clients that are within the organization. For example, the clients may be various departments or employees or other individuals of departments of an enterprise such as the Human Resources department, the Payroll department, etc., or other individuals of the enterprise. In certain other aspects, under a community cloud model, the cloud infrastructure system 602 and the services provided may be shared by several organizations in a related community. Various other models such as hybrids of the above mentioned models may also be used.

Client computing devices 604, 606, and 608 may be of different types (such as devices 502, 504, 506, and 508 depicted in FIG. 5) and may be capable of operating one or more client applications. A user may use a client device to interact with cloud infrastructure system 602, such as to request a service provided by cloud infrastructure system 602.

In some aspects, the processing performed by cloud infrastructure system 602 for providing chatbot services may involve big data analysis. This analysis may involve using, analyzing, and manipulating large data sets to detect and visualize various trends, behaviors, relationships, etc. within the data. This analysis may be performed by one or more processors, possibly processing the data in parallel, performing simulations using the data, and the like. For example, big data analysis may be performed by cloud infrastructure system 602 for determining the intent of an utterance. The data used for this analysis may include structured data (e.g., data stored in a database or structured according to a structured model) and/or unstructured data (e.g., data blobs (binary large objects)).

As depicted in the embodiment in FIG. 6, cloud infrastructure system 602 may include infrastructure resources 630 that are utilized for facilitating the provision of various cloud services offered by cloud infrastructure system 602. Infrastructure resources 630 may include, for example, processing resources, storage or memory resources, networking resources, and the like.

In certain aspects, to facilitate efficient provisioning of these resources for supporting the various cloud services provided by cloud infrastructure system 602 for different tenants, the resources may be bundled into sets of resources or resource modules (also referred to as “pods”). Each resource module or pod may comprise a pre-integrated and optimized combination of resources of one or more types. In certain aspects, different pods may be pre-provisioned for different types of cloud services. For example, a first set of pods may be provisioned for a database service, a second set of pods, which may include a different combination of resources than a pod in the first set of pods, may be provisioned for Java service, and the like. For some services, the resources allocated for provisioning the services may be shared between the services.

Cloud infrastructure system 602 may itself internally use services 632 that are shared by different components of cloud infrastructure system 602 and which facilitate the provisioning of services by cloud infrastructure system 602. These internal shared services may include, without limitation, a security and identity service, an integration service, an enterprise repository service, an enterprise manager service, a virus scanning and whitelist service, a high availability, backup and recovery service, service for enabling cloud support, an email service, a notification service, a file transfer service, and the like.

Cloud infrastructure system 602 may comprise multiple subsystems. These subsystems may be implemented in software, or hardware, or combinations thereof. As depicted in FIG. 6, the subsystems may include a user interface subsystem 612 that enables users of cloud infrastructure system 602 to interact with cloud infrastructure system 602. User interface subsystem 612 may include various different interfaces such as a web interface 614, an online store interface 616 where cloud services provided by cloud infrastructure system 602 are advertised and are purchasable by a consumer, and other interfaces 618. For example, a tenant may, using a client device, request (service request 634) one or more services provided by cloud infrastructure system 602 using one or more of interfaces 614, 616, and 618. For example, a tenant may access the online store, browse cloud services offered by cloud infrastructure system 602, and place a subscription order for one or more services offered by cloud infrastructure system 602 that the tenant wishes to subscribe to. The service request may include information identifying the tenant and one or more services that the tenant desires to subscribe to. For example, a tenant may place a subscription order for a chatbot related service offered by cloud infrastructure system 602. As part of the order, the client may provide information identifying the input (e.g. utterances).

In certain aspects, such as the embodiment depicted in FIG. 6, cloud infrastructure system 602 may comprise an order management subsystem (OMS) 620 that is configured to process the new order. As part of this processing, OMS 620 may be configured to: create an account for the tenant, if not done already; receive billing and/or accounting information from the tenant that is to be used for billing the tenant for providing the requested service to the tenant; verify the tenant information; upon verification, book the order for the tenant; and orchestrate various workflows to prepare the order for provisioning.

Once properly validated, OMS 620 may then invoke the order provisioning subsystem (OPS) 624 that is configured to provision resources for the order including processing, memory, and networking resources. The provisioning may include allocating resources for the order and configuring the resources to facilitate the service requested by the tenant order. The manner in which resources are provisioned for an order and the type of the provisioned resources may depend upon the type of cloud service that has been ordered by the tenant. For example, according to one workflow, OPS 624 may be configured to determine the particular cloud service being requested and identify a number of pods that may have been pre-configured for that particular cloud service. The number of pods that are allocated for an order may depend upon the size/amount/level/scope of the requested service. For example, the number of pods to be allocated may be determined based upon the number of users to be supported by the service, the duration of time for which the service is being requested, and the like. The allocated pods may then be customized for the particular requesting tenant for providing the requested service.

Cloud infrastructure system 602 may send a response or notification 644 to the requesting tenant to indicate when the requested service is now ready for use. In some instances, information (e.g., a link) may be sent to the tenant that enables the tenant to start using and availing the benefits of the requested services.

Cloud infrastructure system 602 may provide services to multiple tenants. For each tenant, cloud infrastructure system 602 is responsible for managing information related to one or more subscription orders received from the tenant, maintaining tenant data related to the orders, and providing the requested services to the tenant or clients of the tenant. Cloud infrastructure system 602 may also collect usage statistics regarding a tenant's use of subscribed services. For example, statistics may be collected for the amount of storage used, the amount of data transferred, the number of users, and the amount of system up time and system down time, and the like. This usage information may be used to bill the tenant. Billing may be done, for example, on a monthly cycle.

Cloud infrastructure system 602 may provide services to multiple tenants in parallel. Cloud infrastructure system 602 may store information for these tenants, including possibly proprietary information. In certain aspects, cloud infrastructure system 602 comprises an identity management subsystem (IMS) 628 that is configured to manage tenant's information and provide the separation of the managed information such that information related to one tenant is not accessible by another tenant. IMS 628 may be configured to provide various security-related services such as identity services, such as information access management, authentication and authorization services, services for managing tenant identities and roles and related capabilities, and the like.

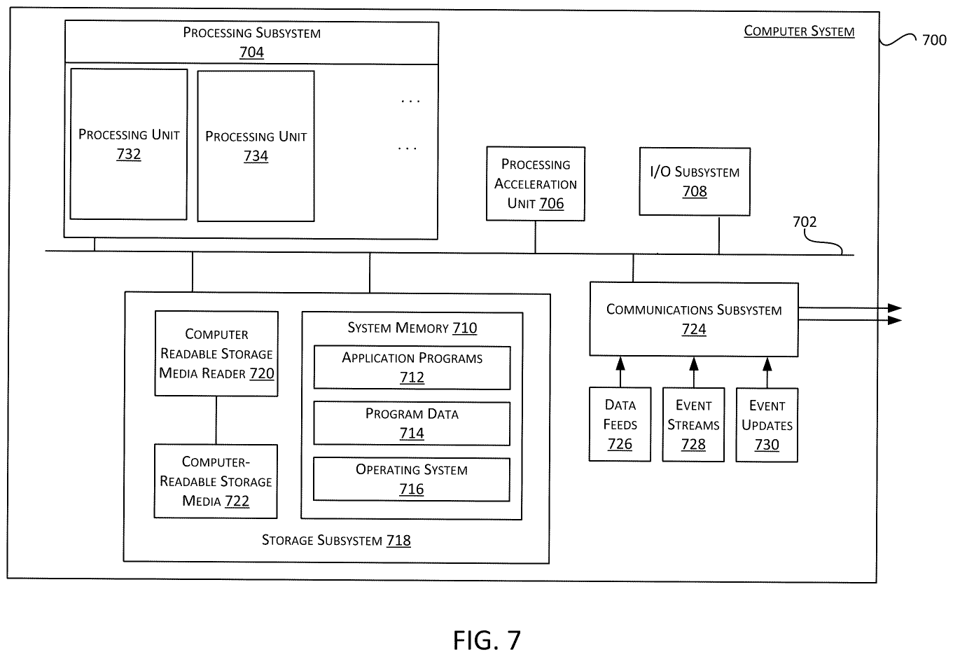

FIG. 7 illustrates an exemplary computer system 700 that may be used to implement certain aspects. As shown in FIG. 7, computer system 700 includes various subsystems including a processing subsystem 704 that communicates with a number of other subsystems via a bus subsystem 702. These other subsystems may include a processing acceleration unit 706, an I/O subsystem 708, a storage subsystem 718, and a communications subsystem 724. Storage subsystem 718 may include non-transitory computer-readable storage media including storage media 722 and a system memory 710.

Bus subsystem 702 provides a mechanism for letting the various components and subsystems of computer system 700 communicate with each other as intended. Although bus subsystem 702 is shown schematically as a single bus, alternative aspects of the bus subsystem may utilize multiple buses. Bus subsystem 702 may be any of several types of bus structures including a memory bus or memory controller, a peripheral bus, a local bus using any of a variety of bus architectures, and the like. For example, such architectures may include an Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, which can be implemented as a Mezzanine bus manufactured to the IEEE P1386.1 standard, and the like.

Processing subsystem 704 controls the operation of computer system 700 and may comprise one or more processors, application specific integrated circuits (ASICs), or field programmable gate arrays (FPGAs). The processors may be single core or multicore processors. The processing resources of computer system 700 can be organized into one or more processing units 732, 734, etc. A processing unit may include one or more processors, one or more cores from the same or different processors, a combination of cores and processors, or other combinations of cores and processors. In some aspects, processing subsystem 704 can include one or more special purpose co-processors such as graphics processors, digital signal processors (DSPs), or the like. In some aspects, some or all of the processing units of processing subsystem 704 can be implemented using customized circuits, such as application specific integrated circuits (ASICs), or field programmable gate arrays (FPGAs).

In some aspects, the processing units in processing subsystem 704 can execute instructions stored in system memory 710 or on computer readable storage media 722. In various aspects, the processing units can execute a variety of programs or code instructions and can maintain multiple concurrently executing programs or processes. At any given time, some or all of the program code to be executed can be resident in system memory 710 and/or on computer-readable storage media 722 including potentially on one or more storage devices. Through suitable programming, processing subsystem 704 can provide various functionalities described above. In instances where computer system 700 is executing one or more virtual machines, one or more processing units may be allocated to each virtual machine.

In certain aspects, a processing acceleration unit 706 may optionally be provided for performing customized processing or for off-loading some of the processing performed by processing subsystem 704 so as to accelerate the overall processing performed by computer system 700.

I/O subsystem 708 may include devices and mechanisms for inputting information to computer system 700 and/or for outputting information from or via computer system 700. In general, use of the term input device is intended to include all possible types of devices and mechanisms for inputting information to computer system 700. User interface input devices may include, for example, a keyboard, pointing devices such as a mouse or trackball, a touchpad or touch screen incorporated into a display, a scroll wheel, a click wheel, a dial, a button, a switch, a keypad, audio input devices with voice command recognition systems, microphones, and other types of input devices. User interface input devices may also include motion sensing and/or gesture recognition devices such as the Meta Quest® controller, Microsoft Kinect® motion sensor, the Microsoft Xbox® 360 game controller, or devices that provide an interface for receiving input using gestures and spoken commands. User interface input devices may also include eye gesture recognition devices such as a blink detector that detects eye activity (e.g., “blinking” while taking pictures and/or making a menu selection) from users and transforms the eye gestures as inputs to an input device. Additionally, user interface input devices may include voice recognition sensing devices that enable users to interact with voice recognition systems (e.g., Siri® navigator or Amazon Alexa®) through voice commands.

Other examples of user interface input devices include, without limitation, three dimensional (3D) mice, joysticks or pointing sticks, gamepads and graphic tablets, and audio/visual devices such as speakers, digital cameras, digital camcorders, portable media players, webcams, image scanners, fingerprint scanners, QR code readers, barcode readers, 3D scanners, 3D printers, laser rangefinders, and eye gaze tracking devices. Additionally, user interface input devices may include, for example, medical imaging input devices such as computed tomography, magnetic resonance imaging, position emission tomography, and medical ultrasonography devices. User interface input devices may also include, for example, audio input devices such as MIDI keyboards, digital musical instruments, and the like.

In general, use of the term output device is intended to include all possible types of devices and mechanisms for outputting information from computer system 700 to a user or other computer. User interface output devices may include a display subsystem, indicator lights, or non-visual displays such as audio output devices, etc. The display subsystem may be any device for outputting a digital picture. Example display devices include flat panel display devices such as those using a light emitting diode (LED) display, a liquid crystal display (LCD) or plasma display, a projection device, a touch screen, a desktop or laptop computer monitor, and the like. As another example, wearable display devices such as Meta Quest® or Microsoft HoloLens® may be mounted to the user for displaying information. User interface output devices may include, without limitation, a variety of display devices that visually convey text, graphics, and audio/video information such as monitors, printers, speakers, headphones, automotive navigation systems, plotters, voice output devices, and modems.

Storage subsystem 718 provides a repository or data store for storing information and data that is used by computer system 700. Storage subsystem 718 provides a tangible non-transitory computer-readable storage medium for storing the basic programming and data constructs that provide the functionality of some aspects. Storage subsystem 718 may store software (e.g., programs, code modules, instructions) that when executed by processing subsystem 704 provides the functionality described above. The software may be executed by one or more processing units of processing subsystem 704. Storage subsystem 718 may also provide a repository for storing data used in accordance with the teachings of this disclosure.

Storage subsystem 718 may include one or more non-transitory memory devices, including volatile and non-volatile memory devices. As shown in FIG. 7, storage subsystem 718 includes a system memory 710 and a computer-readable storage media 722. System memory 710 may include a number of memories including a volatile main random access memory (RAM) for storage of instructions and data during program execution and a non-volatile read only memory (ROM) or flash memory in which fixed instructions are stored. In some implementations, a basic input/output system (BIOS), containing the basic routines that help to transfer information between elements within computer system 700, such as during start-up, may typically be stored in the ROM. The RAM typically contains data and/or program modules that are presently being operated and executed by processing subsystem 704. In some implementations, system memory 710 may include multiple different types of memory, such as static random access memory (SRAM), dynamic random access memory (DRAM), and the like.

By way of example, and not limitation, as depicted in FIG. 7, system memory 710 may load application programs 712 that are being executed, which may include various applications such as Web browsers, mid-tier applications, relational database management systems (RDBMS), etc., program data 714, and an operating system 716. By way of example, operating system 716 may include various versions of Microsoft Windows®, Apple Macintosh®, and/or Linux® operating systems, a variety of commercially-available UNIX® or UNIX-like operating systems (including without limitation the variety of GNU/Linux operating systems, the Oracle Linux®, Google Chrome® OS, and the like) and/or mobile operating systems such as iOS, Windows® Phone, Android® OS, and others.

Computer-readable storage media 722 may store programming and data constructs that provide the functionality of some aspects. Computer-readable media 722 may provide storage of computer-readable instructions, data structures, program modules, and other data for computer system 700. Software (programs, code modules, instructions) that, when executed by processing subsystem 704 provides the functionality described above, may be stored in storage subsystem 718. By way of example, computer-readable storage media 722 may include non-volatile memory such as a hard disk drive, a magnetic disk drive, an optical disk drive such as a CD ROM, digital video disc (DVD), a Blu-Ray® disk, or other optical media. Computer-readable storage media 722 may include, but is not limited to, Zip® drives, flash memory cards, universal serial bus (USB) flash drives, secure digital (SD) cards, DVD disks, digital video tape, and the like. Computer-readable storage media 722 may also include, solid-state drives (SSD) based on non-volatile memory such as flash-memory based SSDs, enterprise flash drives, solid state ROM, and the like, SSDs based on volatile memory such as solid state RAM, dynamic RAM, static RAM, dynamic random access memory (DRAM)-based SSDs, magnetoresistive RAM (MRAM) SSDs, and hybrid SSDs that use a combination of DRAM and flash memory based SSDs.

In certain aspects, storage subsystem 718 may also include a computer-readable storage media reader 720 that can further be connected to computer-readable storage media 722. Reader 720 may receive and be configured to read data from a memory device such as a disk, a flash drive, etc.

In certain aspects, computer system 700 may support virtualization technologies, including but not limited to virtualization of processing and memory resources. For example, computer system 700 may provide support for executing one or more virtual machines. In certain aspects, computer system 700 may execute a program such as a hypervisor that facilitated the configuring and managing of the virtual machines. Each virtual machine may be allocated memory, compute (e.g., processors, cores), I/O, and networking resources. Each virtual machine generally runs independently of the other virtual machines. A virtual machine typically runs its own operating system, which may be the same as or different from the operating systems executed by other virtual machines executed by computer system 700. Accordingly, multiple operating systems may potentially be run concurrently by computer system 700.

Communications subsystem 724 provides an interface to other computer systems and networks. Communications subsystem 724 serves as an interface for receiving data from and transmitting data to other systems from computer system 700. For example, communications subsystem 724 may enable computer system 700 to establish a communication channel to one or more client devices via the Internet for receiving and sending information from and to the client devices. For example, the communications subsystem may be used to transmit a response to a user regarding the inquiry for a chatbot.

Communications subsystem 724 may support both wired and/or wireless communication protocols. For example, in certain aspects, communications subsystem 724 may include radio frequency (RF) transceiver components for accessing wireless voice and/or data networks (e.g., using cellular telephone technology, advanced data network technology, such as 3G, 4G or EDGE (enhanced data rates for global evolution), Wi-Fi (IEEE 802.XX family standards, or other mobile communication technologies, or any combination thereof), global positioning system (GPS) receiver components, and/or other components. In some aspects communications subsystem 724 can provide wired network connectivity (e.g., Ethernet) in addition to or instead of a wireless interface.

Communications subsystem 724 can receive and transmit data in various forms. For example, in some aspects, in addition to other forms, communications subsystem 724 may receive input communications in the form of structured and/or unstructured data feeds 726, event streams 728, event updates 730, and the like. For example, communications subsystem 724 may be configured to receive (or send) data feeds 726 in real-time from users of social media networks and/or other communication services such as Twitter® feeds, Facebook® updates, web feeds such as Rich Site Summary (RSS) feeds, and/or real-time updates from one or more third party information sources.

In certain aspects, communications subsystem 724 may be configured to receive data in the form of continuous data streams, which may include event streams 728 of real-time events and/or event updates 730, that may be continuous or unbounded in nature with no explicit end. Examples of applications that generate continuous data may include, for example, sensor data applications, financial tickers, network performance measuring tools (e.g., network monitoring and traffic management applications), clickstream analysis tools, automobile traffic monitoring, and the like.

Communications subsystem 724 may also be configured to communicate data from computer system 700 to other computer systems or networks. The data may be communicated in various different forms such as structured and/or unstructured data feeds 726, event streams 728, event updates 730, and the like to one or more databases that may be in communication with one or more streaming data source computers coupled to computer system 700.

Computer system 700 can be one of various types, including a handheld portable device (e.g., an iPhone® cellular phone, an iPad® computing tablet, a personal digital assistant (PDA)), a wearable device (e.g., a Meta Quest® head mounted display), a personal computer, a workstation, a mainframe, a kiosk, a server rack, or any other data processing system. Due to the ever-changing nature of computers and networks, the description of computer system 700 depicted in FIG. 7 is intended only as a specific example. Many other configurations having more or fewer components than the system depicted in FIG. 7 are possible. Based on the disclosure and teachings provided herein, a person of ordinary skill in the art can appreciate other ways and/or methods to implement the various aspects.

Although specific aspects have been described, various modifications, alterations, alternative constructions, and equivalents are possible. Embodiments are not restricted to operation within certain specific data processing environments, but are free to operate within a plurality of data processing environments. Additionally, although certain aspects have been described using a particular series of transactions and steps, it should be apparent to those skilled in the art that this is not intended to be limiting. Although some flowcharts describe operations as a sequential process, many of the operations can be performed in parallel or concurrently. In addition, the order of the operations may be rearranged. A process may have additional steps not included in the figure. Various features and aspects of the above-described aspects may be used individually or jointly.

Further, while certain aspects have been described using a particular combination of hardware and software, it should be recognized that other combinations of hardware and software are also possible. Certain aspects may be implemented only in hardware, or only in software, or using combinations thereof. The various processes described herein can be implemented on the same processor or different processors in any combination.

Where devices, systems, components or modules are described as being configured to perform certain operations or functions, such configuration can be accomplished, for example, by designing electronic circuits to perform the operation, by programming programmable electronic circuits (such as microprocessors) to perform the operation such as by executing computer instructions or code, or processors or cores programmed to execute code or instructions stored on a non-transitory memory medium, or any combination thereof. Processes can communicate using a variety of techniques including but not limited to conventional techniques for inter-process communications, and different pairs of processes may use different techniques, or the same pair of processes may use different techniques at different times.

Specific details are given in this disclosure to provide a thorough understanding of the aspects. However, aspects may be practiced without these specific details. For example, well-known circuits, processes, algorithms, structures, and techniques have been shown without unnecessary detail in order to avoid obscuring the aspects. This description provides example aspects only, and is not intended to limit the scope, applicability, or configuration of other aspects. Rather, the preceding description of the aspects can provide those skilled in the art with an enabling description for implementing various aspects. Various changes may be made in the function and arrangement of elements.

The specification and drawings are, accordingly, to be regarded in an illustrative rather than a restrictive sense. It can, however, be evident that additions, subtractions, deletions, and other modifications and changes may be made thereunto without departing from the broader spirit and scope as set forth in the claims. Thus, although specific aspects have been described, these are not intended to be limiting. Various modifications and equivalents are within the scope of the following claims.

What is claimed is:

1. A non-transitory computer-readable medium including instructions that when executed by one or more processors, cause the one or more processors to perform a set of operations including:
accessing a natural-language health record that is generated based on an encounter;
processing the natural-language health record using a natural language processing model, to identify a portion of an extracted text from the natural-language health record;
generating, by applying a binary classification on the portion of the extracted text, a codability of the portion of the extracted text, wherein the codability of the portion of the extracted text is based on whether or an extent to which the portion of the extracted text includes information for assignment of one or more codes;
in response to a positive codability of the portion of the extracted text, assigning two or more codes to the portion of the extracted text by applying a multi-label classification by a machine learning model to the portion of the extracted text;
determining, by applying a probability model, a first probability score indicative of a probability of a combination of the two or more codes being assigned to a single encounter;
determining, by applying a language model, a second probability score that is indicative of the two or more codes assigned to the health record being correct;
assigning, based at least in part on the first probability score and the second probability score, a final probability score to the two or more codes; and
in response to the final probability score being higher than a threshold, causing generation of an insurance record based at least in part on the two or more codes.

2. The non-transitory computer-readable medium of claim 1, wherein to determine the first probability score, the probability model (i) takes into account the assigned two or more codes, and (i) does not take into account the extracted text.

3. The non-transitory computer-readable medium of claim 1, wherein to determine the second probability score, the language model takes into account (i) the assigned two or more codes and (ii) one or both of (a) the extracted text and the (b) health record.

4. The non-transitory computer-readable medium of claim 1, wherein the first probability score is a conditional probability of a first one of the two or more codes being assigned to the single encounter, given that a second one of the two or more codes is assigned to the single encounter.

5. The non-transitory computer-readable medium of claim 1, wherein the probability model is a one of a TF-IDF (term frequency-inverse document frequency) based model, a KNN (k-nearest neighbors) model, or a PMI (probability mutual information) model.

6. The non-transitory computer-readable medium of claim 1, wherein causing generation of the insurance record comprises:
generating one or more billable codes, based on the one or more codes; and
causing generation of the insurance record, based at least in part on the one or more billable codes.

7. The non-transitory computer-readable medium of claim 1, wherein the encounter is a first encounter, the health record is a first health record, the two or more codes are first two or more codes, the final probability score is a first final probability score, the insurance record is a first insurance record, and wherein the set of operations further include:
accessing a second natural-language health record that is generated based on a second encounter;
assigning second one or more codes to the second natural-language health record, by applying a multi-label classification by the machine learning model to another portion of an extracted text of the second natural-language health record;
assigning a second final probability score to the second one or more codes;
in response to the second final probability score lower than the threshold, receiving a correction of at least one of the second one or more codes; and
causing generation of a second insurance record based at least in part on the corrected second one or more codes.

8. The non-transitory computer-readable medium of claim 7, wherein the set of operations further include:
generating training data, based at least in part on the portion of the extracted text of the second natural-language health record and the correction of at least one of the second one or more codes; and
training the machine learning model using the training data.

9. The non-transitory computer-readable medium of claim 1, wherein the set of operations further include:
causing display of an identification of the patient encounter and/or the health record, the two or more codes assigned to the health record, and the final probability score.

10. The non-transitory computer-readable medium of claim 1, wherein accessing the natural-language health record comprises:
pulling the health record from an electronic health record system; or
receiving a push of the health record from the electronic health record system.

11. The non-transitory computer-readable medium of claim 1, wherein preprocessing the natural-language health record comprises:
removing personally identifiable information, such that the extracted natural-language text from the health record lacks any personally identifiable information.

12. The non-transitory computer-readable medium of claim 1, wherein causing generation of the insurance record comprises:
grouping the two or more codes into a single group code;
generating at least one billable code, based at least in part on the single group code; and
causing generation of the insurance record, based at least in part on the at least one billable code.

13. A method comprising:
accessing a natural-language health record that is generated based on an encounter;
processing the natural-language health record using a natural language processing model, to identify a portion of an extracted text from the natural-language health record;
generating, by applying a binary classification on the portion of the extracted text, a codability of the portion of the extracted text, wherein the codability of the portion of the extracted text is based on whether or an extent to which the portion of the extracted text includes information for assignment of one or more codes;
in response to a positive codability of the portion of the extracted text, assigning two or more codes to the portion of the extracted text by applying a multi-label classification by a machine learning model to the portion of the extracted text;
determining, by applying a probability model, a first probability score indicative of a probability of a combination of the two or more codes being assigned to a single encounter;
determining, by applying a language model, a second probability score that is indicative of the two or more codes assigned to the health record being correct;
assigning, based at least in part on the first probability score and the second probability score, a final probability score to the two or more codes; and
in response to the final probability score being higher than a threshold, causing generation of an insurance record based at least in part on the two or more codes.

14. The method of claim 13, wherein to determine the first probability score, the probability model (i) takes into account the assigned two or more codes, and (i) does not take into account the extracted text.

15. The method of claim 13, wherein to determine the second probability score, the language model takes into account (i) the assigned two or more codes and (ii) one or both of (a) the extracted text and the (b) health record.

16. The method of claim 13, wherein the probability model is a one of a TF-IDF (term frequency-inverse document frequency) based model, a KNN (k-nearest neighbors) model, or a PMI (probability mutual information) model.

17. The method of claim 13, wherein causing generation of the insurance record comprises:
grouping the two or more codes into a single group code;
generating at least one billable code, based at least in part on the single group code; and
causing generation of the insurance record, based at least in part on the at least one billable code.

18. A system comprising:
one or more processors; and
one or more non-transitory computer-readable media storing instructions, which, when executed by the system, cause the system to perform operations including:
accessing a natural-language health record that is generated based on an encounter;
processing the natural-language health record using a natural language processing model, to identify a portion of an extracted text from the natural-language health record;
generating, by applying a binary classification on the portion of the extracted text, a codability of the portion of the extracted text, wherein the codability of the portion of the extracted text is based on whether or an extent to which the portion of the extracted text includes information for assignment of one or more codes;
in response to a positive codability of the portion of the extracted text, assigning two or more codes to the portion of the extracted text by applying a multi-label classification by a machine learning model to the portion of the extracted text;
determining, by applying a probability model, a first probability score indicative of a probability of a combination of the two or more codes being assigned to a single encounter;
determining, by applying a language model, a second probability score that is indicative of the two or more codes assigned to the health record being correct;
assigning, based at least in part on the first probability score and the second probability score, a final probability score to the two or more codes; and
in response to the final probability score being higher than a threshold, causing generation of an insurance record based at least in part on the two or more codes.

19. The system of claim 18, wherein:
to determine the first probability score, the probability model (i) takes into account the assigned two or more codes, and (i) does not take into account the extracted text; and
to determine the second probability score, the language model takes into account (i) the assigned two or more codes and (ii) one or both of (a) the extracted text and the (b) health record.

20. The system of claim 18, wherein the first probability score is a conditional probability of a first one of the two or more codes being assigned to the single encounter, given that a second one of the two or more codes is assigned to the single encounter.
